(19) United States
(12) Patent Application Publication
(10) Pub. No.: US 2024/0360431 A1
(43) Pub. Date: Oct. 31, 2024

(54) ENGINEERED ALPHA-GALACTOSIDASE A (A-GAL A) PEPTIDES AND FUNCTIONAL VARIANTS THEREOF AND ASSOCIATED METHODS OF TREATING FABRY DISEASE

(71) Applicant: SICHUAN REAL&BEST BIOTECH CO., LTD., Chengdu (CN)

(72) Inventors: Biao DONG, Chengdu (CN); Siwu HE, Chengdu (CN); Hoi Yee CHOW, Chengdu (CN)

(21) Appl. No.: 18/649,917

(22) Filed: Apr. 29, 2024

Related U.S. Application Data
(60) Provisional application No. 63/499,243, filed on Apr. 30, 2023.
(60) Provisional application No. 63/515,558, filed on Jul. 25, 2023.

(51) Int. Cl. C12N 9/40; A61K 38/00; A61P 3/00; C12N 9/64; C12N 15/86

(52) U.S. Cl. CPC C12N 9/2465; A61P 3/00; C12N 9/6408; C12N 15/86; A61K 38/00; C07K 2319/01; C12N 2750/14143; C12Y 302/01022; C12Y 304/21068

(57) ABSTRACT

Described herein are novel engineered peptides, viral vectors, and pharmaceutical compositions thereof, and uses of the same for increasing α-GAL A expression and for the treatment of conditions associated with α-GAL A deficiency, such as Fabry disease.

### PRIORITY CLAIM

This application claims the benefit of U.S. Provisional Patent Application Nos. 63/499,243, filed Apr. 30, 2023, and 63/515,558, filed Jul. 25, 2023, which are incorporated herein by reference in their entireties.

### SEQUENCE LISTING

This application contains an ST.26 compliant Sequence Listing, which is submitted concurrently in xml format and is hereby incorporated by reference in its entirety. The .xml copy, created on Apr. 26, 2024, is named 149731-8001 US02 Sequence Listing.xml and is 62,000 bytes in size.

### BACKGROUND

Fabry disease is an X-linked lysosomal storage disease characterized by a deficiency of α-galactosidase A (α-GAL A), which may lead to progressive and systemic accumulation of sphingolipids, such as globotriaosylceramide (Gb3) and globotriaosylsphingosine (lyso-Gb3), in the lysosomes of vascular endothelial cells, kidney epithelial cells and endothelial cells, myocardial cells, and more. Deposition of sphingolipids may lead to skin, heart, kidney, cerebrovascular and other diseases, and eventually premature deaths.

Treatments for Fabry disease are limited and include enzyme replacement therapy (ERT), which delivers recombinant human α-GAL A by intravenous injection (J Med Gene 2015 May; 52(5):353-8). Although ERT can be effective, especially in the early stage of the disease, the half-life of α-GAL A is relatively short and patients often require regular infusion (N Engl J Med. 2001 Jul. 5; 345 (1): 9-16), causing a lifelong burden. Recently, the oral pharmacological chaperone, Migalastat®, was approved for the treatment of Fabry disease however, it is only effective for certain α-GAL A mutations. In addition, there are three recombinant adeno-associated virus (rAAV) drugs in clinical trials each using different rAAV serotypes, unique promoters, and other mechanisms for promoting expression of α-GAL A (NCT04046224/NCT05039886, NCT04519749, and NCT04040049). However, rAAV-mediated expression of α-GAL A remains limited as does clearance of disease substrates Gb3 and lyso-Gb3.

Despite these therapies, there is demand for novel safe and effective treatments for Fabry disease having lasting effects.

### SUMMARY

It is an object of the present technology to provide novel compositions for increasing α-GAL A expression and associated methods of treating Fabry disease and/or symptoms of Fabry disease using such novel compositions.

Unless specified otherwise, α-GAL A refers to the mature amino acid sequence after the wildtype signal peptide (wt sp) of α-GAL A is cleaved, or functional variants thereof; a precursor or full length α-GAL A refers to α-GAL A fused to one or more signal peptides, which may be wt sp or a heterologous sp, e.g., without limitation, sp1, sp2, . . . , sp22 disclosed in Table 2; hGLA refers to a nucleotide sequence that encode α-GAL A, e.g., the wildtype human α-galactosidase A (wt hGLA) and optimized/modified nucleotide sequence that encodes α-GAL A or functional variant thereof, e.g., hGLA c.o. v1 disclosed herein; lowercase sp refers to a signal peptide or functional variant thereof while upper case SP refers to a nucleotide sequence that encodes a signal peptide or functional variant thereof. The signal peptide referred to by sp may be the wt sp or one of the heterologous sp's, and nucleotide sequence SP that encodes the signal peptide may be a nucleotide sequence SP encoding the wt sp or one of the heterologous sp's.

In some embodiments, the compositions comprise an engineered peptide comprising α-GAL A or a functional variant thereof, and signal peptide of tissue plasminogen activator (sp21) or a functional variant thereof, and optionally a cell-penetrating peptide or functional variant thereof. In some embodiments, the compositions comprise a viral vector comprising an expression cassette comprising a first nucleotide sequence encoding α-GAL A or the functional variant thereof, a second nucleotide sequence encoding sp21 or the functional variant thereof, and optionally a third nucleotide sequence encoding the cell-penetrating peptide or the functional variant thereof, and a promoter.

In some embodiments, the compositions comprise an engineered peptide comprising α-GAL A or a functional variant thereof, wt signal peptide of α-GAL A (wt sp) or a functional variant thereof, and optionally a cell-penetrating peptide or functional variant thereof. In some embodiments, the compositions comprise a viral vector comprising an expression cassette comprising a first nucleotide sequence encoding α-GAL A or the functional variant thereof, a fourth nucleotide sequence encoding wt sp or the functional variant thereof, and optionally a third nucleotide sequence encoding the cell-penetrating peptide or the functional variant thereof, and a promoter.

It is another object of the present technology to provide methods of treating Fabry disease or reducing one or more symptoms of Fabry disease in a subject by administering the compositions to the subject.

It is another object of the present technology to provide methods of increasing α-GAL A expression in one or more target tissues of a subject by administering the compositions to the subject.

It is another object of the present technology to provide methods of reducing sphingolipid storage in one or more target tissues of a subject by administering the compositions to the subject.

These and other objects, which will become apparent during the following detailed description and accompanying drawings, have been achieved by the present technology which includes engineered peptides comprising or consisting of a portion of α-GAL A or a functional variant thereof and at least a portion of an heterologous signal peptide and viral vectors encoding the same which unexpectedly yield beneficial therapeutic activities through increased expression, secretion, localization, and activity to treat, reduce, or otherwise ameliorate Fabry disease.

In some embodiments, the engineered peptide comprises α-GAL A or a functional variant thereof and a heterologous signal peptide (e.g., sp21) or a functional variant thereof. Other examples of the heterologous signal peptides include, without limitation, sp1-sp20, and sp22. In certain embodiments, the heterologous signal peptides are selected from the group consisting of sp1, sp3, sp4, sp18, sp20, and sp22 as disclosed herein. α-GAL A or the functional variant thereof may or may not comprise the wild type (wt) signal peptide of α-GAL A.

In some aspects, the engineered peptide further comprises a cell-penetrating peptide or a functional variant thereof. Examples of the cell-penetrating peptide include, without limitation, TAT p47-57, TAT p48-60, Penetratin p43-58, hCT p9-32, polyarginines (R7-R25), pVEC, Pep-1, Transportan, and MAP.

In some aspects, α-GAL A or the functional variant thereof comprises a peptide sequence with at least about 60%, 65%, 70%, 75%, 80%, 85%, 90%, 95%, 99%, or 100% identity to SEQ ID NO: 27.

In some aspects, sp1, sp3, sp4, sp18, sp20, sp21 and sp22, or the functional variant thereof comprises a peptide sequence with at least about 60%, 65%, 70%, 75%, 80%, 85%, 90%, 95%, 99%, or 100% identity to SEQ ID NOs: 29, 31, 32, 46, 48, 49 and 50, respectively.

In some aspects, the signal peptide or the functional variant thereof comprises a peptide sequence with at least about 60%, 65%, 70%, 75%, 80%, 85%, 90%, 95%, 99%, or 100% identity to a peptide sequence selected from the group consisting of SEQ ID NOs: 29, 31, 32, 46, 48, 49 and 50.

In some aspects, the cell-penetrating peptide or the functional variant thereof comprises a peptide sequence with at least about 60%, 65%, 70%, 75%, 80%, 85%, 90%, 95%, 99%, or 100% identity to a peptide sequence selected from the group consisting of TAT p47-57 (SEQ ID NO: 55), TAT p48-60 (SEQ ID NO: 56), Penetratin p43-58 (SEQ ID NO: 57), hCT p9-32 (SEQ ID NO: 58), polyarginines (SEQ ID NO: 59), pVEC (SEQ ID NO: 60), Pep-1 (SEQ ID NO: 61), Transportan (SEQ ID NO: 62), and MAP (SEQ ID NO: 63).

In some aspects, the heterologous signal peptide (e.g., sp21) or the functional variant thereof is fused to the N terminus of the portion of α-GAL A or the functional variant thereof. In some aspects, the portion of the heterologous signal peptide (e.g., sp21) or the functional variant thereof is fused to the C terminus of the portion of α-GAL A or the functional variant thereof. In some aspects, the portion of the heterologous signal peptide (e.g., sp21) or the functional variant thereof is fused within the portion of α-GAL A or the functional variant thereof.

In some aspects, the cell-penetrating peptide or the functional variant thereof is fused to the C terminus of the portion of α-GAL A or the functional variant thereof. In some aspects, the cell-penetrating peptide or the functional variant thereof is fused to the N terminus of the portion of α-GAL A or the functional variant thereof. In some aspects, the cell-penetrating peptide or the functional variant thereof is fused within the portion of α-GAL A or the functional variant thereof.

In some embodiments, the present technology further comprises viral vectors having a nucleotide sequence encoding the engineered peptides.

In some aspects, the viral vectors further comprise a promoter.

In some embodiments, the compositions of the present technology comprise a viral vector comprising an expression cassette comprising or consisting of a first nucleotide sequence encoding the portion of α-GAL A or the functional variant thereof, and a second nucleotide sequence encoding the portion of the heterologous signal peptide (e.g., sp21) or the functional variant thereof. In some embodiments, the compositions of the present technology comprise a viral vector comprising an expression cassette comprising or consisting of a first nucleotide sequence encoding the portion of α-GAL A or the functional variant thereof, a fourth nucleotide sequence encoding the portion of wt sp or the functional variant thereof, and optionally a third nucleotide sequence encoding the portion of the cell-penetrating peptide or the functional variant thereof, and a promoter.

In some aspects, the second or fourth nucleotide sequence is 5′ end to the first nucleotide sequence and/or wherein the third nucleotide sequence is 3′ end to the first nucleotide sequence.

Another aspect described herein is the viral vector wherein the nucleotide sequence encoding the portion of α-GAL A or the functional variant thereof has a nucleotide sequence with at least about 60%, 65%, 70%, 75%, 80%, 85%, 90%, 95%, 99%, or 100% identity to SEQ ID NO: 25 or 26.

In some aspects, the nucleotide sequence encoding the portion of sp21 or the functional variant thereof has a nucleotide sequence with at least about 60%, 65%, 70%, 75%, 80%, 85%, 90%, 95%, 99%, or 100% identity to SEQ ID NO: 22.

In some aspects, the nucleotide sequence encoding the portion of wt sp or the functional variant thereof has a nucleotide sequence with at least about 60%, 65%, 70%, 75%, 80%, 85%, 90%, 95%, 99%, or 100% identity to SEQ ID NO: 1 or 51.

In some aspects, the nucleotide sequence encoding the portion of the portion of the heterologous signal peptide or the functional variant thereof has a nucleotide sequence with at least about 60%, 65%, 70%, 75%, 80%, 85%, 90%, 95%, 99%, or 100% identity to a nucleotide sequence selected from the group consisting of SEQ ID NOs: 2, 3, 4,5,6,7,8,9,10,11,12,13,14,15,16,17,18,19,20,21, 22, 23, 64, 65, and 66.

In some aspects, the nucleotide sequence encoding the portion of the portion of the heterologous signal peptide or the functional variant thereof has a nucleotide sequence with at least about 60%, 65%, 70%, 75%, 80%, 85%, 90%, 95%, 99%, or 100% identity to a nucleotide sequence selected from the group consisting of SEQ ID NOs: 2, 4, 5, 19, 21, 22, and 23.

In some aspects, the nucleotide sequence encoding the portion of cell-penetrating peptide or the functional variant thereof has a nucleotide sequence with at least about 60%, 65%, 70%, 75%, 80%, 85%, 90%, 95%, 99%, or 100% identity to SEQ ID NO: 24.

In some aspects, the nucleotide sequence encoding the portion of cell-penetrating peptide or the functional variant thereof has a nucleotide sequence with at least about 60%, 65%, 70%, 75%, 80%, 85%, 90%, 95%, 99%, or 100% identity to a nucleotide sequence selected from the group consisting of SEQ ID NOs: 24, 52, 53, and 54.

In some aspects, the promoter is a tissue-specific promoter.

In some aspects, the tissue-specific promoter is a liver-specific promoter, a heart specific promoter, or a kidney specific promoter.

In some aspects, the liver-specific promoter is a TTR promoter.

In some aspects, the viral vector further comprises at least one inverted terminal repeat (ITR) nucleotide sequence, an intron, and a polyadenylation (polyA) nucleotide sequence.

In some aspects, the viral vector is a modified recombinant adeno-associated viral (rAAV) vector.

In some embodiments, the present technology further comprises methods of treating Fabry disease or reducing one or more symptoms of Fabry disease in a subject by administering the compositions to the subject.

In some embodiments, the present technology further comprises methods of increasing α-GAL A expression in one or more target tissues of a subject by administering the compositions to the subject.

In some embodiments, the present technology further comprises methods of reducing sphingolipid storage in one or more target tissues of a subject by administering the compositions to the subject.

In some aspects, the subject has Fabry disease or has symptoms of Fabry disease.

In some aspects, the one or more target tissues are selected from the group consisting of a liver tissue, a heart tissue, or a kidney tissue.

In some aspects, the methods further comprise administration of the compositions of the present technology to the subject.

In some aspects, the compositions of the present technology are administered to the subject once.

In some aspects, the compositions of the present technology are administered to the subject via systemic administration.

In some aspects, the compositions of the present technology are administered to the subject via local administration.

In some aspects, the compositions of the present technology are administered to the subject via tissue specific administration.

In some aspects, the viral vector of compositions comprising the viral vector of the present technology are administered to the subject in one or more doses of 1 E+11 vg/kg to 5E+14 vg/kg.

### BRIEF DESCRIPTION OF THE DRAWINGS

FIGS. 1A and 1B illustrate embodiments of engineered recombinant adeno-associated virus (rAAV) vectors of the present technology. The rAAV vectors comprise an expression construct having a human α-galactosidase A (hGLA) nucleotide sequence and a nucleotide sequence encoding a signal peptide (sp). The hGLA nucleotide sequence may be the wild type hGLA (hGLA wt) or an embodiment of optimized nucleotide sequence encoding α-GAL A (hGLA c.o. v1). The signal peptide nucleotide sequence may be the wild type nucleotide sequence encoding the wild type sp of α-GAL A (wt SP), optimized nucleotide sequence encoding the wild type signal peptide of α-GAL A (wtSP c.o.), or nucleotide sequences encoding heterologous sp, e.g., sp1 to sp22as disclosed herein (e.g., SP1 to SP22, respectively). Embodiments of rAAV vectors comprising nucleotide sequences encoding wt sp (wt SP, wtSP c.o.-1, wtSP c.o.-2, wtSP c.o.-3 or wtSP c.o.) or sp21 (SP21) are illustrated in FIGS. 1A and 1B. FIG. 1A shows two embodiments of single-stranded rAAV vectors (ss-rAAVs): ss-wtspGLA comprising wt SP and hGLA wt; and ss-sp21GLA comprising SP21 and hGLA wt. FIG. 1B shows multiple embodiments of self-complimentary vectors (sc-rAAVs): FD802-1 comprising wt SP and hGLA wt; FD802-2 comprising SP21 and hGLA wt; FD802-3 comprising wtSP c.o. and hGLA c.o. v1; FD802-4 comprising SP21 and hGLA c.o. v1; FD802-5 comprising wtSP c.o., hGLA c.o. v1 and a nucleotide sequence (tat) which is the wild type nucleotide sequence that encodes TAT (e.g., TAT sequence (p47-57), TAT sequence (p48-60)).

FIGS. 2A and 2B depict increased α-GAL A expression (FIG. 2A) and activity levels (FIG. 2B) in media removed from HepG2 cell culture vehicles from HepG2 cells transfected with plasmids expressing α-GAL A+heterologous sp (sp1, sp2, . . . , and sp22), relative to cells transfected with plasmids expressing α-GAL A+wt sp.

FIGS. 3A-B depict the genomic distribution of the ss-rAAV vectors (FIG. 3A) and the hGLA mRNA expression (FIG. 3B) in the liver of Fabry mice injected with low (2E+12 vg/kg), medium (5E+12 vg/kg), or high (5E+13 vg/kg) dose of an embodiment of ss-rAAV expressing α-GAL A+sp21 (ss-sp21 GLA), or an embodiment of ss-rAAV expressing α-GAL A+wt sp (ss-wtspGLA), or untreated Fabry mice (0 vg/kg dose).

FIG. 4 illustrates increased α-GAL A activity in plasma of Fabry mice injected with low (2E+12 vg/kg), medium (5E+12 vg/kg), or high (5E+13 vg/kg) dose of an embodiment of ss-rAAV expressing α-GAL A+sp21 (ss-sp21GLA), or an embodiment of ss-rAAV expressing α-GAL A+wt sp (ss-wtspGLA) compared to untreated Fabry mice (Untreated) and untreated wildtype mice (Wild type).

FIGS. 5A-D demonstrate increased α-GAL A activity in the liver (FIG. 5A), heart (FIG. 5B), kidney (FIG. 5C), and spleen (FIG. 5D) of Fabry mice injected with low (2E+12 vg/kg), medium (5E+12 vg/kg), or high (5E+13 vg/kg) dose of an embodiment of ss-rAAV expressing α-GAL A+sp21 (ss-sp21 GLA), relative to Fabry mice injected with low (2E+12 vg/kg), medium (5E+12 vg/kg), or high (5E+13 vg/kg) dose of an embodiment of ss-rAAV expressing α-GAL A+wt sp (ss-wtspGLA), untreated Fabry mice (Untreated), and untreated wildtype mice (Wild type).

FIGS. 6A-B illustrate increased expression of α-GAL A in the liver of Fabry mice 12-weeks following injection with low (2E+12 vg/kg), medium (5E+12 vg/kg), or high (5E+13 vg/kg) dose of an embodiment of ss-rAAV expressing α-GAL A+sp21 (ss-sp21 GLA), relative to mice injected with an embodiment of ss-rAAV expressing α-GAL A with wt sp (ss-wtspGLA)(FIG. 6A), and increased level of glycosylated of α-GAL A under high dose (5E+13 vg/kg) of the embodiment of ss-rAAV expressing α-GAL A+sp21 (ss-sp21GLA), relative to mice injected with high dose (5E+13 vg/kg) of the embodiment of ss-rAAV expressing α-GAL A+wt sp (ss-wtspGLA) (FIG. 6B). Recombinant α-GAL A was used as a positive control for glycosylation test in vitro (Rec.hAGA).

FIG. 7 illustrates increased expression of α-GAL A in the liver, heart, and kidney of Fabry mice 12-weeks following injection with high dose (5E+13 vg/kg) of the embodiment of ss-rAAV expressing α-GAL A+sp21 (ss-sp21GLA), relative to Fabry mice injected with high dose (5E+13 vg/kg) of the embodiment of ss-rAAV expressing α-GAL A+wt sp (ss-wtspGLA) or untreated Fabry mice.

FIGS. 8A-B show reduction of lyso-Gb3 in the plasma of Fabry mice following injection with low (2E+12 vg/kg), medium (5E+12 vg/kg), or high (5E+13 vg/kg) dose of an embodiment of ss-rAAV expressing α-GAL A+sp21 (ss-sp21GLA), relative to Fabry mice injected with low (2E+12 vg/kg), medium (5E+12 vg/kg), or high (5E+13 vg/kg) dose of an embodiment of ss-rAAV expressing α-GAL A+wt sp (ss-wtspGLA) and untreated Fabry mice at both 12-weeks following injection (FIG. 8A) and 26-weeks following injection (FIG. 8B).

FIG. 9 depicts the recorded body weight of Fabry mice following injection with low (2E+12 vg/kg), medium (5E+12 vg/kg), or high (5E+13 vg/kg) dose of an embodiment of ss-rAAV expressing α-GAL A+sp21 (ss-sp21GLA) or an embodiment of ss-rAAV expressing α-GAL A+wt sp (ss-wtspGLA), in addition to untreated Fabry mice (Untreated) and wild type mice (Wild type).

FIG. 10 illustrates liver tissue of Fabry mice following injection with high dose (5E+13 vg/kg) of an embodiment of ss-rAAV expressing α-GAL A+sp21 (ss-sp21GLA) or an embodiment of ss-rAAV expressing α-GAL A+wt sp (ss-wtspGLA), untreated Fabry mice, and wild type mice without Fabry disease. No obvious histopathological changes in the liver after high-dose administrations of ss-wtspGLA and ss-sp21 GLA administration, indicating that the therapeutic administration has a certain degree of safety.

FIG. 11 demonstrates increased α-GAL A expression in HepG2 protein lysates following transfection with a plasmid expressing α-GAL A encoded by a functional variant hGLA nucleotide sequence (hGLA c.o. v1, SEQ ID NO: 26) relative to cells transfected with a plasmid expressing α-GAL A encoded by a wt hGLA nucleotide sequence (hGLA wt, SEQ ID NO: 25) and cells without transfection as negative controls (NC). GAPDH works as the loading control of the Western Blotting.

FIG. 12 depicts increased α-GAL A activity in plasma of Fabry mice following injection with low (2E+12 vg/kg) or medium (5E+12 vg/kg) dose of embodiments of sc-rAAV vectors expressing α-GAL A encoded by functional variant hGLA nucleotide sequence hGLA c.o. v1 (FD802-3 & FD802-4) and relative to Fabry mice injected with low (2E+12 vg/kg) or medium (5E+12 vg/kg) dose of embodiments of sc-rAAV vectors expressing α-GAL A encoded by a wt hGLA nucleotide sequence (FD802-1 & FD802-2), or low (2E+12 vg/kg) or medium (5E+12 vg/kg) dose of an embodiment of ss-rAAV expressing α-GAL A+sp21 (ss-sp21 GLA), and untreated Fabry mice (Untreated).

FIG. 13 depicts increased α-GAL A activity in the liver, heart, kidney, and spleen of Fabry mice following injection with medium dose (5E+12 vg/kg) of FD802-1, FD802-2, FD802-3, and FD802-4 relative to Fabry mice injected with medium dose (5E+12 vg/kg) of an embodiment of ss-rAAV expressing α-GAL A+sp21 (ss-sp21GLA), and untreated Fabry mice (Untreated).

FIG. 14 illustrates increased detection of α-GAL A in the liver samples of Fabry mice following injection with medium dose (5E+12 vg/kg) of FD802-1, FD802-2, FD802-3, and FD802-4 relative to Fabry mice injected with medium dose (5E+12 vg/kg) of an embodiment of ss-rAAV expressing α-GAL A+sp21 (ss-sp21 GLA), and untreated Fabry mice(Untreated).

FIG. 15 depicts increased detection of α-GAL A in the liver of Fabry mice following injection with medium dose (5E+12 vg/kg) of FD802-1, FD802-2, FD802-3, and FD802-4 relative to Fabry mice injected with medium dose (5E+12 vg/kg) of an embodiment of ss-rAAV expressing α-GAL A+sp21 (ss-sp21GLA), and untreated Fabry mice.

FIG. 16 illustrates increased level of glycosylated α-GAL A in the liver of Fabry mice following injection with medium dose (5E+12 vg/kg) of FD802-3 and FD802-4 relative to Fabry mice injected with medium dose (5E+12 vg/kg) of FD802-1 or FD802-2. Recombinant α-GAL A was used as a positive control for glycosylation in vitro (Rec.hAGA).

FIGS. 17A-B depict reduced lyso-Gb3 in the plasma of Fabry mice following injection with medium dose (5E+12 vg/kg) of FD802-1, FD802-2, FD802-3, and FD802-4 relative to Fabry mice injected with medium dose (5E+12 vg/kg) of an embodiment of ss-rAAV expressing α-GAL A+sp21 (ss-sp21 GLA), and untreated Fabry mice (Untreated) at both 6-weeks following injection (FIG. 17A) and 14-weeks following injection (FIG. 17B).

FIG. 18 illustrates tissue histopathological staining of liver of Fabry mice following injection with medium dose (5E+12 vg/kg) of FD802-1, FD802-2, FD802-3, and FD802-4 relative to Fabry mice injected with medium dose (5E+12 vg/kg) of an embodiment of ss-rAAV expressing α-GAL A+sp21 (ss-sp21 GLA), and untreated Fabry mice (Untreated). There were no obvious histopathological changes in the liver after administration, indicating that the therapeutic administration has a certain degree of safety.

FIGS. 19A-B demonstrate the correlation between vector serotypes (rAAV1, rAAv2, . . . , rAAV10) and α-GAL A expression in (Lysate) in and secretion from (Media) HepG2 cells (FIG. 19A) following transducing various serotypes sc-rAAV with the genome of FD802-4, which comprises a functional variant hGLA nucleotide sequence (hGLA c.o. v1, SEQ ID NO: 26) and SP21 (SEQ ID NO: 22). FIG. 19B shows activity analysis of the secreted α-GAL A. NC: Negative control, a protein sample of HepG2 cells that were not transduced with AAV. PC: positive control, a protein sample containing α-GAL A.

FIGS. 20A-B illustrate the inclusion of a nucleotide sequence encoding TAT p47-57 in an α-GAL A-expressing plasmid and its impact on α-GAL A activity in cell culture. FIG. 20A shows that FD802-5 (Ctat-GLAco) produced a lower activity level of α-GAL A in culture media when compared to FD802-3 (GLAco). FIG. 20B shows that the α-GAL A protein expressed from both plasmids had comparable enzyme activities.

FIGS. 21A-B depict increased α-GAL A activity in the plasma of Fabry mice following injection with low (2E+12 vg/kg) or medium (5E+12 vg/kg) dose of FD802-5 relative to untreated mice, but no change in activity when compared to comparable doses of FD802-3 (FIG. 21A). Activity levels are also depicted in the liver, heart, kidney, and spleen tissues of Fabry mice following injection with medium dose (5E+12 vg/kg) of FD802-5 compared to both medium dose (5E+12 vg/kg) injection of FD802-3 and untreated mice (Untreated) (FIG. 21B).

FIG. 22 illustrates increased detection of α-GAL A in the liver, heart and kidney in the Fabry mice following injection with medium dose (5E+12 vg/kg) of FD802-5 relative to comparable doses of FD802-3 and untreated mice (Untreated).

FIGS. 23A-B show α-GAL A expression (FIG. 23A) and glycosylation (FIG. 23B) in the liver of Fabry mice 14 weeks following injection with medium dose (5E+12 vg/kg) of FD802-3 and FD802-5. Recombinant α-GAL A was used as a positive control for glycosylation test in vitro (Rec.hAGA).

FIGS. 24A-B depict reduced lyso-Gb3 in plasma of Fabry mice at both 6 weeks (FIG. 24A) and 14 weeks (FIG. 24B) following injection with low (2E+12 vg/kg) or medium (5E+12 vg/kg) dose of FD802-3 and FD802-5 relative to untreated mice (Untreated), with FD802-5 showing a dose-dependent effect.

FIG. 25 illustrates no visible negative impact to the liver following injection with medium dose FD802-5 relative to untreated Fabry mice and Fabry mice treated with medium dose FD802-3.

### DETAILED DESCRIPTION

Provided herein are compositions useful for the treatment of Fabry disease and/or alleviating symptoms of Fabry disease.

Regular infusion of recombinant α-GAL A, termed enzyme replacement therapy (ERT), is currently the primary treatment option for Fabry patients with non-amenable mutations, whereas patients with amenable mutations can benefit from both ERT and treatment using small molecule chaperones. However, recombinant α-GAL A has low physical stability, a short circulating half-life, and variable uptake into different disease-relevant tissues, which may limit the efficacy of ERT. The compositions provided herein deliver nucleic acid sequences encoding recombinant α-GAL A that are effective for gene therapy and permit longer duration of circulating enzyme activity, prior to α-GAL A being taken up into the target tissues.

In certain embodiments, the compositions and methods described herein include nucleic acid sequences, expression cassettes, vectors, recombinant viruses, peptide sequences, engineered peptides, and other compositions and methods for expression of a functional α-GAL A. In certain embodiments, the compositions and methods described herein include nucleic acid sequences, expression cassettes, vectors, recombinant viruses, peptide sequences, engineered peptides, host cells, other compositions and methods for production of a composition comprising either a nucleic acid sequence encoding a functional α-GAL A. In yet another embodiment, the compositions and methods described herein include nucleic acid sequences, expression cassettes, vectors, recombinant viruses, peptide sequences, engineered peptides, and other compositions and methods for delivery of the nucleic acid sequence encoding a functional α-GAL A to a subject for the treatment of Fabry disease. In one embodiment, the compositions and methods described herein are useful for providing therapeutic levels of functional α-GAL A in the periphery, such as, for example, in the blood, liver, kidney, and/or peripheral nervous system of a subject. In certain embodiments, an adeno-associated viral (AAV) vector-based method described herein provides a new treatment option, helping to restore a desired function of α-GAL A and to alleviate a symptom associated with α-GAL A deficiency and/or dysfunction (Fabry disease) by providing expression of functional α-GAL A in a subject in need thereof.

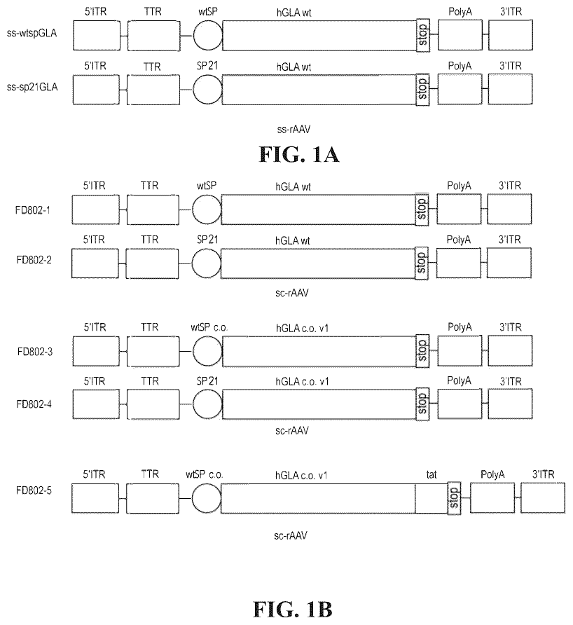

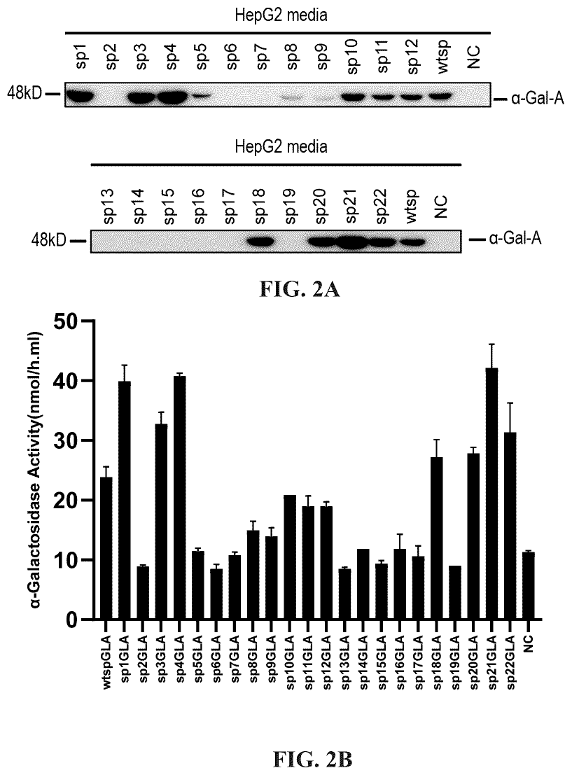

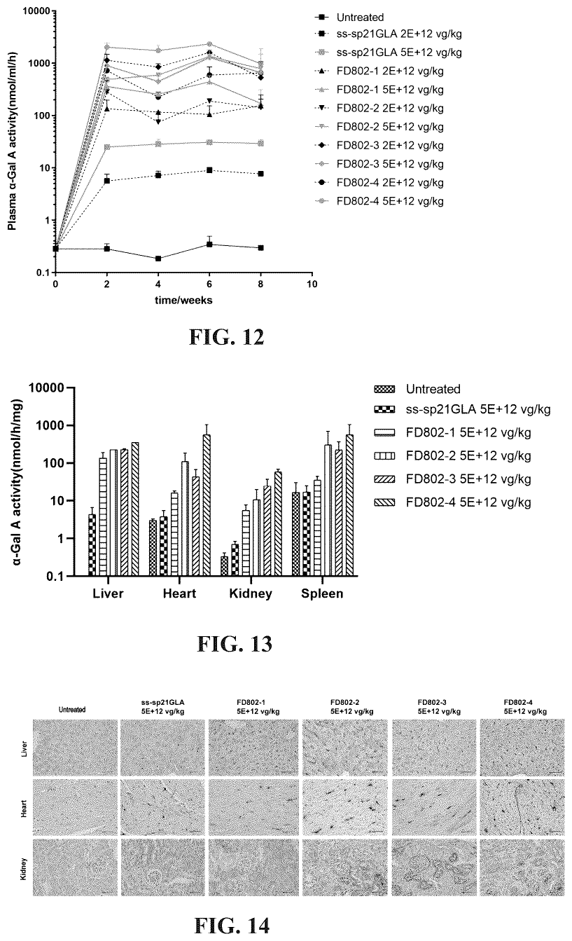

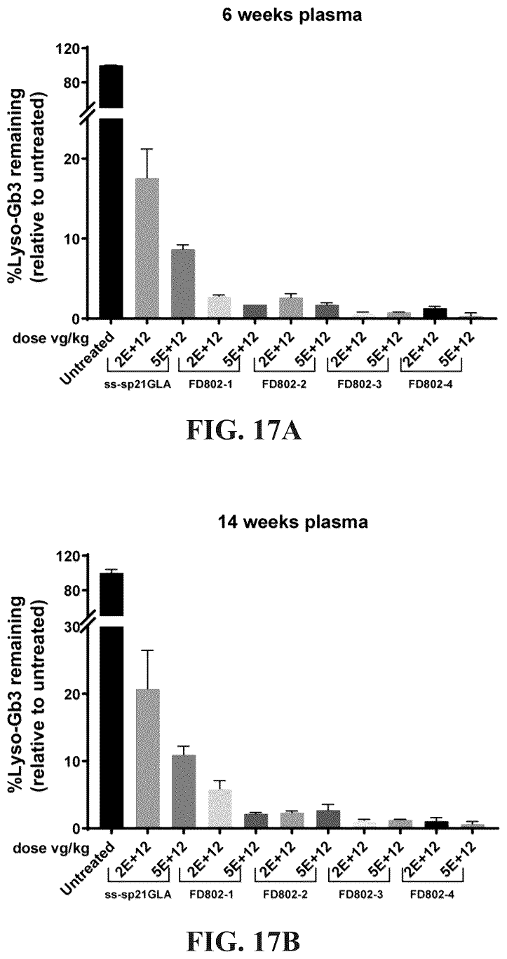

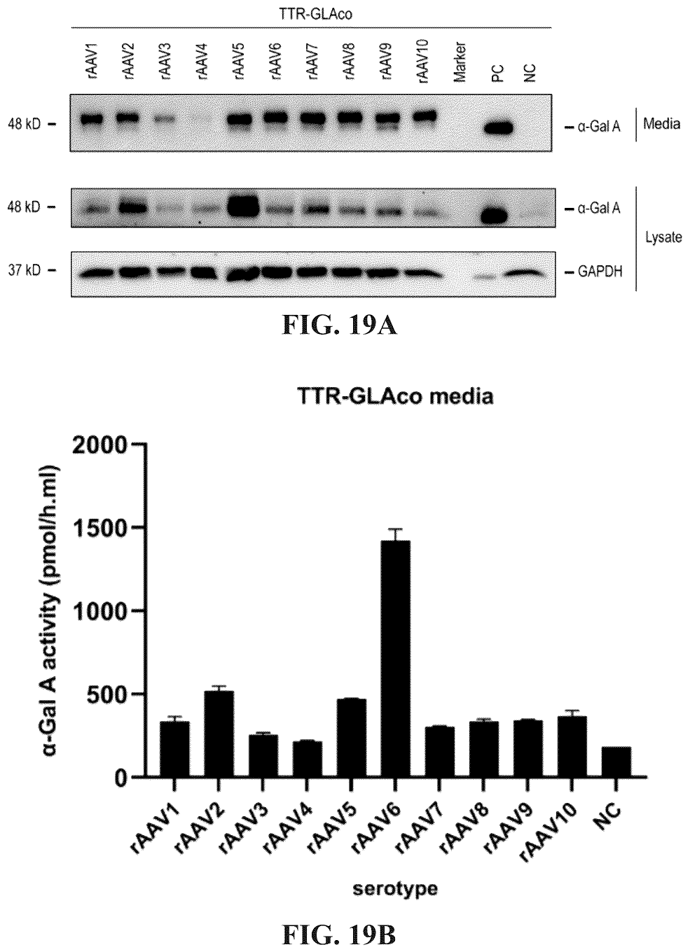

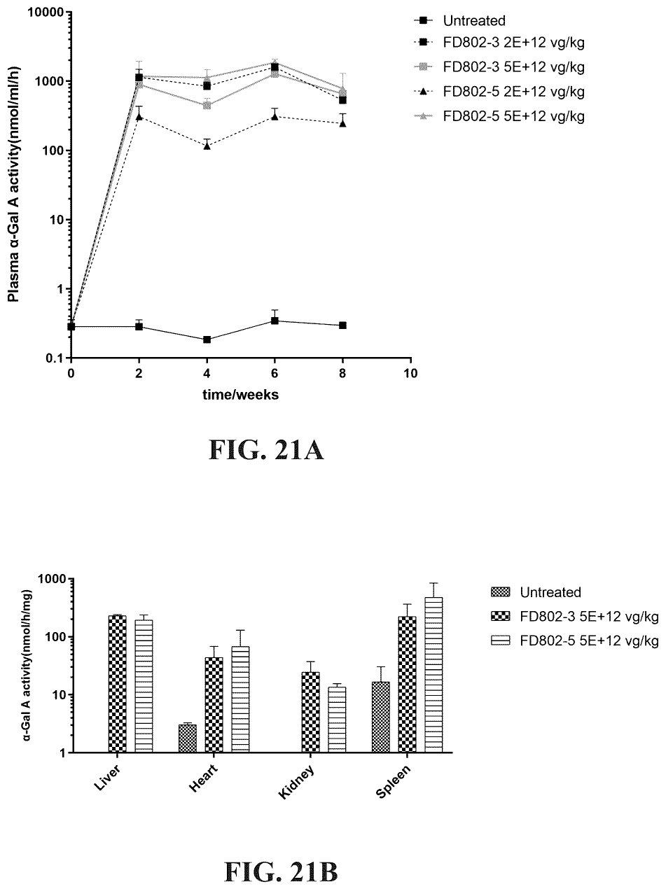

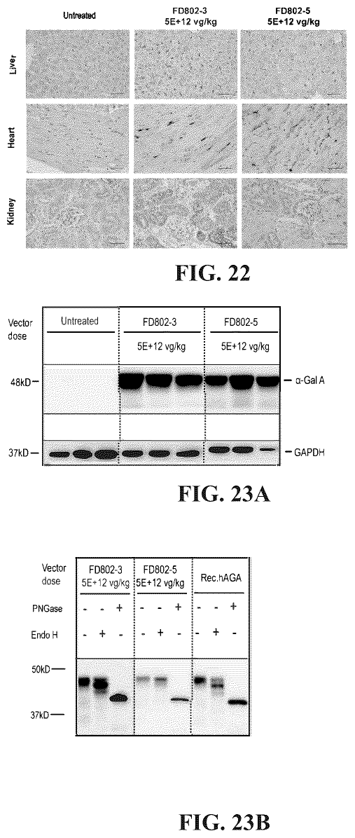

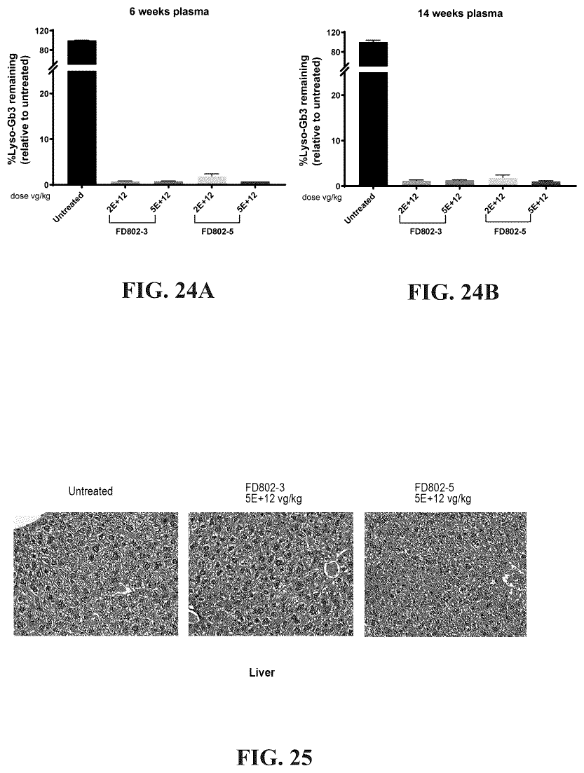

As shown in the Example section, several heterologous signal peptides (sp1-sp22) were screened in vitro for comparable or better extracellular secretion of α-GAL A, and sp1, 3, 4, 18, and 20-22 showed improved extracellular secretion of α-GAL A compared to wt sp (Example 1, FIGS. 2A & 2B). Human-origin sp21 was selected as an example of heterologous sp to construct rAAV vectors comprising nucleotide sequences encoding α-GAL A fused with heterologous sp (FIGS. 1A & 1B, vectors ss-sp21GLA, FD802-2, and FD802-4) to compare with rAAV vectors expressing α-GAL A fused with wt sp (FIGS. 1A & 1B, vectors ss-wtspGLA, FD802-1, and FD802-3). Fabry mice were treated with rAAV vectors to compare α-GAL A expression and activities as well as lyso-Gb3 clearance in plasma, various organs and/or tissues. Data show that heterologous sp such as sp21 was superior to wt sp in α-GAL A expression and secretion, as well as lyso-Gb3 clearance without perturbing tissue structures in various examples. Examples 1-3 tested rAAV vectors comprising wt hGLA and nucleotide sequence encoding a sp (wt sp or sp21) in Fabry mice, and the sp21 group showed improved α-GAL A expression and activities in plasma, liver, heart, kidney and spleen, and improved lyso-Gb3 clearance in plasma with less weight gain. Examples 4-5 tested rAAV vectors comprising an optimized hGLA nucleotide sequence (hGLA c.o.v.1) and sp nucleotide sequence (wt SP, wtSP c.o., or SP21) in Fabry mice, and the hGLA c.o.v.1 groups showed improved α-GAL A expression and activities in plasma (FIG. 12), liver, heart, kidney and spleen (FIG. 13), and improved lyso-Gb3 clearance in plasma (FIGS. 17A & 17B) than the corresponding wt hGLA groups. Example 6 shows that α-GAL A expression of FD802-4 vector comprising hGLA c.o.v.1 and SP21 were successful in various rAAV serotype other than rAAV8 (FIGS. 19A & 19B). Examples 7 and 8 tested rAAV vectors comprising hGLA c.o.v.1 and a functional variant of the wt SP nucleotide (wtSP c.o.), and with or without a nucleotide sequence encoding TAT p47-57 (tat). rAAV vector comprising hGLA c.o.v.1, wtSP c.o. and tat (FD802-5) showed higher α-GAL A activities in heart and spleen, while lower α-GAL A activities in liver, kidney (FIG. 21B) and plasma (FIG. 21A) compared to rAAV vector comprising hGLA c.o.v.1 and wtSP c.o but without tat (FD802-3) when administered at 5E+12 vg/kg to Fabry mice. Improved α-GAL A protein levels were observed in the heart in Fabry mice injected with FD802-5 compared to FD802-3 injected Fabry mice (FIG. 22). Fusion of TAT p47-57 to α-GAL A did not appear to alter liver expression or glycosylation of α-GAL A in Fabry mice (FIGS. 23A-B), and vectors expressing α-GAL A with TAT p47-57 (FD802-5) also showed effective lyso-Gb3 reduction (FIGS. 24A-B) and no observable negative effects on the liver tissue in Fabry mice (FIG. 25).

### Definitions

Unless defined otherwise, all technical and scientific terms used herein have the same meaning as commonly understood by those of ordinary skill in the art to which the present technology belongs. For the purposes of the present technology, the following terms are defined below.

The articles “a” and “an” are used herein to refer to one or to more than one (i.e., to at least one) of the grammatical object of the article. Byway of example, “an element” means one element or more than one element. Likewise, any reference to singular includes plural embodiments, and any reference to more than one component may include a singular embodiment.

The term “about” means a quantity, level, value, number, frequency, percentage, dimension, size, amount, weight or length that varies by acceptable levels in the art. In some embodiments, such variation may be as much as 30, 25, 20, 15, 10, 9, 8, 7, 6, 5, 4, 3, 2 or 1% to a reference quantity, level, value, number, frequency, percentage, dimension, size, amount, weight or length. When the term “about” is used in conjunction with a numerical range, it modifies that range by extending the boundaries above and below the numerical values set forth.

As used herein, the term “a therapeutic level” or “therapeutically effective” of a functional α-GAL A means an enzyme activity of at least about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, about 100%, more than 100%, about 2-fold, about 3-fold, or about 5-fold of a healthy control. Suitable assays for measuring α-GAL A enzymatic activity are known to those of skill in the art. In some embodiments, such therapeutic levels of α-GAL A may result in alleviation of Fabry disease-related symptoms; improvement of Fabry disease-related biomarkers of disease (for example, reduction of Gb3 levels in serum, urine and/or other biological samples); facilitation of other treatments for Fabry disease (e.g., enzyme replacement or chaperone therapy); prevention of neurocognitive decline; reversal of certain Fabry disease-related symptoms and/or prevention of progression of Fabry disease-related symptoms; or any combination thereof.

As used herein, “a healthy control” refers to a subject or a biological sample therefrom, wherein the subject does not have Fabry disease or α-GAL A deficiency and/or dysfunction otherwise. The healthy control can be from one subject. In another embodiment, the healthy control is a pooled sample from multiple subjects.

As used herein, the term “biological sample” refers to any cell, biological fluid, or tissue. Suitable samples for use in this invention may include, without limitation, whole blood, leukocytes, fibroblasts, serum, urine, plasma, saliva, bone marrow, cerebrospinal fluid, amniotic fluid, and skin cells. Such samples may further be diluted with saline, buffer, or a physiologically acceptable diluent. Alternatively, such samples are concentrated by conventional means.

It is intended that each of the vectors and other compositions described herein may be useful in another embodiment. In addition, it is also intended that each of the compositions described as useful in the methods may be an embodiment of the invention.

As used herein, “disease,” “disorder,” and “condition” refer to Fabry disease and/or α-GAL A deficiency and/or dysfunction in a subject.

As used herein, “engineered” refers to the construction of a peptide or vector to comprise specific peptide or genetic elements, such as a specific nucleotide sequence to produce a certain protein product. This also comprises the manipulation of both endogenous and exogenous genetic or peptide elements to elicit a desired product, nucleic acid or protein sequence, and/or structure.

As used herein, “portion” refers to a nucleotide sequence or amino acid sequence that is at least about 60%, 65%, 70%, 75%, 80%, 85%, 90%, 95%, 99%, or 100% of the full-size genetic element or peptide. For example, a portion of an hGLA nucleotide sequence comprises a nucleotide sequence that is at least about 60%, 65%, 70%, 75%, 80%, 85%, 90%, 95%, 99%, or 100% of the full hGLA nucleotide sequence. Another example is that a portion of an α-GAL A comprises an amino acid sequence that is at least about 60%, 65%, 70%, 75%, 80%, 85%, 90%, 95%, 99%, or 100% of the full α-GAL A.

As used herein “wildtype,” “wt,” and “endogenous” are used interchangeably to describe a naturally occurring or innate gene, protein, nucleotide sequence, or peptide sequence.

As used herein, “heterologous” is used to describe a non-naturally occurring or extrinsic gene, protein, nucleotide sequence, or peptide sequence.

As used herein, “a nucleic acid” refers to a polymeric form of nucleotides and includes RNA, mRNA, cDNA, genomic DNA, peptide nucleic acid (PNA) and synthetic forms and mixed polymers of the above. A nucleotide refers to a ribonucleotide, deoxynucleotide or a modified form of either type of nucleotide (e.g., a peptide nucleic acid oligomer). The term also includes single- and double-stranded forms of DNA. The skilled person will appreciate that functional variants of these nucleic acid molecules are described herein. Functional variants are nucleic acid sequences that can be directly translated, using the standard genetic code, to provide an amino acid sequence identical to that translated from a parental nucleic acid molecule.

In certain embodiments, the nucleic acid molecules encoding a functional α-GAL A, and other constructs as described herein are useful in generating expression cassettes and vector genomes and may be engineered for expression in yeast cells, insect cells, or mammalian cells, such as human cells. Methods are known and have been described previously, such as those described in WIPO Patent Application Publication Number WO 1996/09378, which is incorporated herein by reference in its entirety. A nucleotide sequence is considered engineered if at least one non-preferred codon, as compared to a wt sequence, is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in www.kazusa.jp/codon. Preferably more than one non-preferred codon, preferably most or all nonpreferred codons, are replaced by codons that are more preferred. Preferably the most frequently used codons in an organism are used in an engineered nucleotide sequence. Replacement by preferred codons generally leads to higher expression. It will also be understood by a skilled person that numerous different nucleic acid molecules can encode the same polypeptide as a result of the degeneracy of the genetic code. It is also understood that skilled persons may, using routine techniques, make nucleotide substitutions that do not affect the amino acid sequence encoded by the nucleic acid molecules to reflect the codon usage of any particular host organism in which the polypeptides are to be expressed. Therefore, unless otherwise specified, a “nucleic acid sequence encoding an amino acid sequence” and “a nucleotide sequence encoding an amino acid sequence” includes all nucleotide sequences that are degenerate versions of each other and that encode the same amino acid sequence. Nucleic acid sequences can be cloned using routine molecular biology techniques, or generated de novo by DNA synthesis, which can be performed using routine procedures by service companies having business in the field of DNA synthesis and/or molecular cloning (e.g., GeneArt, GenScript, Life Technologies, Eurofins).

In certain embodiments, the nucleic acids, expression cassettes, vector genomes described herein include an α-GAL A-coding nucleotide sequence that is an engineered nucleotide sequence. In certain embodiments, the engineered sequence is useful to improve production, transcription, expression, or safety in a subject. In certain embodiments, the engineered sequence is useful to increase efficacy of the resulting therapeutic compositions or treatment. In further embodiments, the engineered nucleotide sequence is useful to increase the efficacy of the functional α-GAL A being expressed and may also permit a lower dose of a therapeutic reagent that encode the functional α-GAL A. In certain embodiments, the engineered hGLA nucleotide coding sequence is characterized by an improved translation rate as compared to a wild type hGLA nucleotide coding sequence. By “engineered” is meant that the nucleic acid sequences encoding a functional α-GAL A described herein are assembled and placed into any suitable genetic element, e.g., naked DNA, phage, transposon, cosmid, episome, etc., which transfers hGLA nucleotide sequences carried thereon to a host cell, e.g., for generating non-viral delivery systems (e.g., RNA-based systems, naked DNA, or the like), or for generating viral vectors in a packaging host cell, and/or for delivery to a host cell in a subject. In certain embodiments, the genetic element is a vector. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

The term “percent (%) identity,” “sequence identity,” “percent sequence identity,” or “percent identical” in the context of nucleic acid sequences refers to the residues in the two nucleotide sequences which are the same when aligned for correspondence. The length of sequence identity comparison may be over the full-length of a construct, the full-length of a gene coding nucleotide sequence, or a fragment of at least about 500 to 1,000 nucleotides. However, identity among smaller fragments, for example, of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 100 amino acids, about 300 amino acids, or a peptide fragment thereof or the corresponding nucleic acid sequence coding sequences. A suitable amino acid fragment may be at least about 8 amino acids in length and may be up to about 50 amino acids. Generally, when referring to “identity,” “homology,” or “similarity” between two different amino acid sequences, “identity,” “homology” or “similarity” is determined in reference to “aligned” amino acid sequences. “Aligned” amino acid sequences or “alignments” refer to multiple nucleic acid sequences or protein (amino acids) sequences, often containing corrections for missing or additional bases or amino acids as compared to a reference amino acid sequence.

Identity may be determined by preparing an alignment of amino acid sequences and using a variety of algorithms and/or computer programs known in the art or commercially available (e.g., BLAST, ExPASy; Clustal Omega; FASTA; using, e.g., Needleman-Wunsch algorithm, Smith-Waterman algorithm). Alignments are performed using any of a variety of publicly or commercially available Multiple Sequence Alignment Programs. Sequence alignment programs are available for amino acid sequences, e.g., the “Clustal Omega,” “Clustal X,” “MAP,” “PIMA,” “MSA,” “BLOCKMAKER,” “MEME,” and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments,” 27(13):2682-2690 (1999).

As used herein, the terms “human GLA,” “hGLA,” and “GLA” are used interchangeably to refer to the therapeutic gene that encodes the mature α-GAL A or functional variant thereof. It will be understood that the Greek letter “alpha” and the symbol “a” are used interchangeably throughout this specification. Examples of hGLA include, without limitation, native (wildtype) hGLA that encode the mature α-GAL A, functional variants of the wt hGLA, and optimized nucleotide sequences thereof (e.g., hGLA c.o. v1). In some embodiments, hGLA comprises or consists of SEQ ID NO: 25. In some aspects, hGLA comprises or consists of a nucleotide sequence that has at least about 60%, 65%, 70%, 75%, 80%, 85%, 90%, 95%, 99%, or 100% identity to SEQ ID NO: 25. In certain aspects, hGLA comprises or consists of a nucleotide sequence that has at least about 60%, 65%, 70%, 75%, 80%, 85%, 90%, 95%, 99%, or 100% identity to SEQ ID NO: 25. In certain aspects, hGLA comprises or consists of a nucleotide sequence that has about 60%, 65%, 70%, 75%, 80%, 85%, 90%, 95%, 99%, or 100% identity to SEQ ID NO: 25. In some embodiments, hGLA comprises or consists of SEQ ID NO: 26. In some aspects, hGLA comprises or consists of a nucleotide sequence that has at least about 60%, 65%, 70%, 75%,80%, 85%, 90%, 95%, 99%, or 100% identity to SEQ ID NO: 26. In certain aspects, hGLA comprises or consists of a nucleotide sequence that has at least about 60%, 65%, 70%, 75%, 80%, 85%, 90%, 95%, 99%, or 100% identity to SEQ ID NO: 26. In certain aspects, hGLA comprises or consists of a nucleotide sequence that has about 60%, 65%, 70%, 75%, 80%, 85%, 90%, 95%, 99%, or 100% identity to SEQ ID NO: 26.

As used herein, the term “α-GAL A” is used to refer to a human alpha galactosidase A protein, or the therapeutic peptide. Alternative names for alpha galactosidase A include agalsidase alfa, alpha-D-galactosidase A, alpha galactosidase A protein, alpha galactosidase A enzyme, alpha-D-galactoside galactohydrolase, alpha-galactosidase, alphα-galactosidase A, ceramidetrihexosidase, GALA, α-GAL A, galactosidase, alpha galactosidase, and melibiase. It will be understood that the Greek letter “alpha” and the symbol “a” are used interchangeably throughout this specification. Examples of α-GAL A Include, without limitation, native (wildtype) α-GAL A and, in particular, variants of α-GAL A expressed from the nucleic acid sequences provided herein, or functional fragments thereof, which restore a desired function, ameliorate symptoms, improve symptoms associated with a Fabry disease-related biomarker (e.g., serum α-GAL A), and/or facilitate other treatment(s) for Fabry disease when delivered in a composition or by a method as provided herein.

The “α-GAL A,” unless specified otherwise, refers to the mature protein after the signal peptide is cleaved, a variant protein as described herein, or a functional fragment. As used herein, the term “functional α-GAL A” refers to an enzyme having the amino acid sequence of the full-length native (wildtype) protein (as shown in UniProtKB accession number: P06280-1), a variant thereof (including those described herein with specific amino acid substitution(s)), a mutant thereof with a conservative amino acid replacement, a fragment thereof, a full-length or a fragment of any combination of the variant and the mutant with a conservative amino acid replacement, which provides at least about 10%, at least about 20%, at least about 30%, at least about 40%, at least about 50%, at least about 60%, at least about 65%, at least about 70%, at least about 75%, at least about 80%, at least about 90%, or about the same, or greater than 100% of the biological activity level of a native (wild-type) α-GAL A. In some embodiments, α-GAL A encoded by a nucleotide sequence comprising or consisting of SEQ ID NO: 25. In some aspects, α-GAL A is encoded by a nucleotide sequence that has at least about 60%, 65%, 70%, 75%, 80%, 85%, 90%, 95%, 99%, or 100% identity to SEQ ID NO: 25. In certain aspects, α-GAL A is encoded by a nucleotide sequence that has at least 60%, 65%, 70%, 75%, 80%, 85%, 90%, 95%, 99%, or 100% identity to SEQ ID NO: 25. In certain aspects, α-GAL A is encoded by a nucleotide sequence that has about 60%, 65%, 70%, 75%, 80%, 85%, 90%, 95%, 99%, or 100% identity to SEQ ID NO: 25. In some embodiments, α-GAL A is encoded by a nucleotide sequence comprising or consisting of SEQ ID NO: 26. In some aspects, α-GAL A is encoded by a nucleotide sequence that has at least about 60%, 65%, 70%, 75%, 80%, 85%, 90%, 95%, 99%, or 100% identity to SEQ ID NO: 26. In certain aspects, α-GAL A is encoded by a nucleotide sequence that has at least 60%, 65%, 70%, 75%, 80%, 85%, 90%, 95%, 99%, or 100% identity to SEQ ID NO: 26. In certain aspects, α-GAL A is encoded by a nucleotide sequence that has about 60%, 65%, 70%, 75%, 80%, 85%, 90%, 95%, 99%, or 100% identity to SEQ ID NO: 26.

As used herein, a “functional variant” of a peptide or nucleotide sequence refers to a protein or nucleotide variant of the peptide or nucleotide sequence (including those described herein with specific amino acid and nucleotide substitution(s)), a mutant thereof with a conservative amino acid or nucleotide replacement, a fragment thereof, a full-length or a fragment of any combination of the variant and the mutant with a conservative amino acid or nucleotide replacement, which provides at least about 10%, at least about 20%, at least about 30%, at least about 40%, at least about 50%, at least about 60%, at least about 65%, at least about 70%, at least about 75%, at least about 80%, at least about 90%, or about the same, or greater than 100% of the biological activity level or expression abilities of the peptide (e.g., a wt α-GAL A) or nucleotide sequence (e.g., a wt hGLA).

As used herein, “signal peptide” or “sp” refers to a peptide sequence fused to or within a therapeutic peptide that influences function or localization of the therapeutic peptide. The signal peptide (sp) can be fused to or near the N terminus of the therapeutic peptide, fused to or near the C terminus of the therapeutic peptide, or within the therapeutic peptide, or any combinations thereof. The sp can be wildtype or heterologous in nature. Lowercase sp refers to a signal peptide or functional variant thereof while upper case SP refers to a nucleotide sequence that encodes a signal peptide or functional variant thereof. A nucleotide sequence that encodes an sp may be referred to as SP. The signal peptide referred to by sp may be the wt sp or one of the heterologous sp's disclosed herein, and nucleotide sequence SP that encodes the signal peptide may be a nucleotide sequence SP encoding the wt sp or one of the heterologous sp's.

As used herein, “tissue-specific promoter” refers to a genetic element which stimulates expression of a peptide sequence in a predefined tissue or tissues. The tissue-specific promoter can stimulate expression of the peptide sequence in one or more than one tissue.

As used herein, “vector” refers to a nucleotide sequence which introduces an exogenous or engineered nucleotide sequence into a cell. The vector can be a linear or circular element. The vector may be, for example, a plasmid, virus, phage, or cosmid. The vector may comprise of one or more than one expression cassettes. The vector may be engineered.

As used herein, “viral vector” refers to the use of a viral genome to deliver genetic material to a cell. The viral genome can be modified for the inclusion or exclusion of genetic elements. The viral vector may be designed or synthesized in vivo or in vitro. The viral vector may be a retrovirus, lentivirus, poxvirus, adenovirus, and adeno-associated virus. The viral vector may also be single-stranded or self-complimentary, such as an ss-rAAV or an sc-rAAV, respectively.

As used herein, “serotype” refers to the biologically distinguishing features and/or variation of a particle, such as the surface antigens. The serotype may be selected from at least one of rAAV1, rAAV2, rAAV3, rAAV4, rAAV5, rAAV6, rAAV7, rAAV8, rAAV9, rAAV10, rAAV11, rAAV12, rAAV13, and any derivatives thereof, e.g., engineered using directed evolution.

As used herein, “cell-penetrating peptide” refers to a peptide, protein, or protein fragment that can cross cell or organelle membranes or that has the ability to transport cargo across cell or organelle membranes. The cell-penetrating peptide can be fused to or near the N terminus of the therapeutic peptide, fused to or near the C terminus of the therapeutic peptide, or within the therapeutic peptide, or any combinations thereof.

As used herein, “encapsidated” and “encapsidation” refers to the packaging, securing, or enclosure of a vector with structural proteins and/or lipid molecules.

As used herein, “Gb3” refers to the sphingolipid, glycolipid globotriaosylceramide. Gb3 can also be known as “GIs” or “GL3.” Gb3 can be a free sphingolipid or conjoined to a different molecule or cellular membrane. Gb3 also may refer to the acetylated version of Gb3 or acetylated Lyso-Gb3.

As used herein, “lyso-Gb3,” refers to the deacetylated version of Gb3. Lyso-Gb3 also refers to a plasma biomarker of Fabry disease. Lyso-Gb3 can also be known as “lyso-GIs” or “lyso-GL3.”

As used herein, “patient” or “subject” refers to a male or female mammal, e.g., human, dogs, and animal models used for clinical research. In certain embodiments, the subject of these methods and compositions is a human diagnosed with Fabry disease. In further embodiments, the human subject of these methods and compositions is a prenatal, a newborn, an infant, a toddler, a preschool-aged child, a grade-school-aged child, a teen, a young adult, or an adult.

As used herein, “sphingolipid storage” refers to the processing involving anabolism and catabolism of sphingolipids and the distribution of sphingolipids in leukocytes and tissues including, but not limited to, bone marrow, skin, and muscle. Sphingolipid storage may also refer to α-GAL A activity. Sphingolipid storage disease can refer to α-GAL A deficiency or dysfunction, or the absence of enzymes involved in sphingolipid metabolism.

As used herein, the term “Fabry disease,” “Fabry-related symptom(s)” or “symptom(s)” refers to symptom(s) found in patients with Fabry disease as well as in animal models for Fabry disease. Such symptoms include but are not limited to angiokeratomas, acroparesthesia, hypohidrosis/anhidrosis, corneal, lenticular opacity, cardiac problems, pain, and a reduction in kidney function. Further, common cardiac-related signs and symptoms of Fabry disease include left ventricular hypertrophy, valvular disease (especially mitral valve prolapse and/or regurgitation), premature coronary artery disease, angina, myocardial infarction, conduction abnormalities, arrhythmias, congestive heart failure. Fabry disease is referred to by other names including alphα-galactosidase A deficiency, Anderson-Fabry disease, and angiokeratoma corporis diffusum.

As used herein, “Fabry mouse” refers to a mouse model deficient in α-GAL A and may be used as a model of Fabry disease. For example, doi: 10.1073/pnas.94.6.2540, and see Proc Natl Acad Sci USA. 1997 Mar. 18; 94 (6): Proc Natl Acad Sci USA. 1997 Mar. 18; 94 (6): 2540-42540-4.

As used herein, “administering,” “administer,” and “administration” refer to delivery of therapies or compositions of the present technology to a subject either by local or systemic administration. Administration may be intratracheal, intranasal, epidermal, transdermal, oral, or parenteral. Parenteral administration includes intravenous, intraarterial, subcutaneous, intraperitoneal or intramuscular injection or infusion; or intracranial, e.g., intrathecal or intraventricular, administration.

As used herein, “therapeutically effective amount” refers to an amount that produces a desired effect in a subject for an indication, condition, disease, or disorder. In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including, but not limited to, the characteristics of the therapeutic composition (e.g., activity, pharmacokinetics, pharmacodynamics, and bioavailability); the physiological condition of the subject (e.g., age, body weight, sex, disease type and stage, medical history, general physical condition, responsiveness to a given dosage, and other present medications); the nature of any pharmaceutically acceptable carriers, excipients, and preservatives in the composition; and the route of administration. One skilled in the clinical and pharmacological arts will be able to determine a therapeutically effective amount through routine experimentation, namely, by monitoring a subject's response to administration of the therapeutic composition and adjusting the dosage accordingly.

As used herein, “clinically effective amount,” “clinically effective concentration,” or “clinically effective dose” refers to a concentration or dose of a peptide, composition, or pharmaceutical composition that is shown to be effective in clinical trials or is predicted to be effective based on early phase or pre-clinical trials. In some embodiments, a “clinically effective amount” is the same as a “therapeutically effective amount.” In some embodiments, a “clinically effective amount” is higher or lower than a “therapeutically effective amount.” For additional guidance, see Remington: The Science and Practice of Pharmacy, 21st Edition, Univ. of Sciences in Philadelphia (USIP), Lippincott Williams & Wilkins, Philadelphia, PA, 2005.

### Engineered Peptide

Described herein are engineered peptides designed to deliver α-GAL A to a cell or subject in need thereof. α-GAL A catalyzes the hydrolysis of sphingolipids and is involved in glycosphingolipid degradation in the lysosome. In the absence or dysfunction of α-GAL A, sphingolipids, such as glycosphingolipids, and their byproducts may accumulate to harmful levels. This accumulation and α-GAL A deficiency is a commonly observed phenomenon in subjects with Fabry disease. Therapies which promote increased α-GAL A activity are sought out for Fabry disease treatment. One potential therapy is delivering α-GAL A to a subject via engineered peptides. Restoring α-GAL A levels using engineered peptides may promote the metabolic homeostasis, specifically in the instance of sphingolipid metabolism. Engineered peptides comprising α-GAL A may be further modified to increase efficiency, targeting, and stability of the peptide. Modifications may include alteration of the wt α-GAL A amino acid sequence to generate functional variants, which in turn may be more effective in restoring α-GAL A function than wt α-GAL A amino acid sequences.

In some embodiments, α-GAL A is human α-GAL A. In some embodiments, the engineered peptide comprises α-GAL A or a functional variant thereof. In some embodiments, the engineered peptide further comprises one or more signal peptides or functional variants thereof, and optionally one or more cell-penetrating peptides or functional variants thereof. The one or more signal peptides or functional variants thereof as well as the one or more cell-penetrating peptides or functional variants thereof may be fused to or near C and/or N terminus of α-GAL A or the functional variant thereof, and/or fused internally within the sequence of α-GAL A or the functional variant thereof. In certain embodiments, the engineered peptide comprises a spacer sequence between α-GAL A or the functional variant thereof and the one or more signal peptides or functional variants. In certain embodiments, the engineered peptide comprises a spacer sequence between α-GAL A or the functional variant thereof and the one or more cell-penetrating peptides or functional variants. In certain embodiments, the engineered peptide comprises at least one signal peptides fused at or close to the N terminus of α-GAL A or the functional variant thereof. In certain embodiments, the engineered peptide comprises multiple signal peptides. The multiple signal peptides may be fused sequentially together at N terminus. In certain embodiments, the engineered peptide comprises multiple cell-penetrating peptides. The multiple cell-penetrating peptides may be fused sequentially together at any sites of the protein.

In certain aspects, the human α-GAL A is encoded by wt hGLA (SEQ ID NO: 25). In certain aspects, the human α-GAL A is encoded by a functional variant of a wt nucleotide sequence. In certain aspects, the functional variant is hGLA c.o. v1 (SEQ ID NO: 26).

In some embodiments, the engineered peptide comprises α-GAL A or the functional variant thereof that comprises a peptide sequence with at least about 60% to 100% identity to SEQ ID NO: 27. In some embodiments, the engineered peptide comprises α-GAL A or the functional variant thereof that comprises a peptide sequence with at least about 60%, 65%, 70%, 75%, 80%, 85%, 90%, 95%, 99%, or 100% identity to SEQ ID NO: 27.

In some embodiments, the engineered peptide comprises α-GAL A or the functional variant thereof that comprises a peptide sequence with at least 60% to 100% identity to SEQ ID NO: 27. In some embodiments, the engineered peptide comprises α-GAL A or the functional variant thereof that comprises a peptide sequence with at least 60%, 65%, 70%, 75%, 80%, 85%, 90%, 95%, 99%, or 100% identity to SEQ ID NO: 27.

In some embodiments, the engineered peptide comprises α-GAL A or the functional variant thereof that comprises a peptide sequence with about 60% to 100% identity to SEQ ID NO: 27. In some embodiments, the engineered peptide comprises α-GAL A or the functional variant thereof that comprises a peptide sequence with about 60%, 65%, 70%, 75%, 80%, 85%, 90%, 95%, 99%, or 100% identity to SEQ ID NO: 27.

TABLE 1
Amino acid sequence of α-GAL A and embodiments of nucleotide sequences
encoding α-GAL A.

| SEQUENCE | | |
|---|---|---|
| NAME | SEQUENCE | SEQ ID NO. |
| hGLA wt | CTGGACAATGGATTGGCAAGGACGCCTACCAT | 25 |
| | GGGCTGGCTGCACTGGGAGCGCTTCATGTGC | |
| | AACCTTGACTGCCAGGAAGAGCCAGATTCCTG | |
| | CATCAGTGAGAAGCTCTTCATGGAGATGGCAG | |
| | AGCTCATGGTCTCAGAAGGCTGGAAGGATGCA | |
| | GGTTATGAGTACCTCTGCATTGATGACTGTTGG | |
| | ATGGCTCCCCAAAGAGATTCAGAAGGCAGACT | |
| | TCAGGCAGACCCTCAGCGCTTTCCTCATGGGA | |
| | TTCGCCAGCTAGCTAATTATGTTCACAGCAAAG | |
| | GACTGAAGCTAGGGATTTATGCAGATGTTGGA | |
| | AATAAAACCTGCGCAGGCTTCCCTGGGAGTTT | |
| | TGGATACTACGACATTGATGCCCAGACCTTTG | |
| | CTGACTGGGGAGTAGATCTGCTAAAATTTGAT | |
| | GGTTGTTACTGTGACAGTTTGGAAAATTTGGCA | |
| | GATGGTTATAAGCACATGTCCTTGGCCCTGAAT | |
| | AGGACTGGCAGAAGCATTGTGTACTCCTGTGA | |
| | GTGGCCTCTTTATATGTGGCCCTTTCAAAAGCC | |
| | CAATTATACAGAAATCCGACAGTACTGCAATCA | |
| | CTGGCGAAATTTTGCTGACATTGATGATTCCTG | |
| | GAAAAGTATAAAGAGTATCTTGGACTGGACATC | |
| | TTTTAACCAGGAGAGAATTGTTGATGTTGCTGG | |
| | ACCAGGGGGTTGGAATGACCCAGATATGTTAG | |
| | TGATTGGCAACTTTGGCCTCAGCTGGAATCAG | |
| | CAAGTAACTCAGATGGCCCTCTGGGCTATCAT | |
| | GGCTGCTCCTTTATTCATGTCTAATGACCTCCG | |
| | ACACATCAGCCCTCAAGCCAAAGCTCTCCTTC | |
| | AGGATAAGGACGTAATTGCCATCAATCAGGAC | |
| | CCCTTGGGCAAGCAAGGGTACCAGCTTAGACA | |
| | GGGAGACAACTTTGAAGTGTGGGAACGACCTC | |
| | TCTCAGGCTTAGCCTGGGCTGTAGCTATGATA | |
| | AACCGGCAGGAGATTGGTGGACCTCGCTCTTA | |
| | TACCATCGCAGTTGCTTCCCTGGGTAAAGGAG | |
| | TGGCCTGTAATCCTGCCTGCTTCATCACACAG | |
| | CTCCTCCCTGTGAAAAGGAAGCTAGGGTTCTA | |
| | TGAATGGACTTCAAGGTTAAGAAGTCACATAAA | |
| | TCCCACAGGCACTGTTTTGCTTCAGCTAGAAAA | |
| | TACAATGCAGATGTCATTAAAAGACTTACTTTA | |
| | A | |
| hGLA c.o. v1 | CTGGACAACGGCCTGGCCAGAACCCCCACCAT | 26 |
| | GGGCTGGCTGCACTGGGAGAGATTCATGTGCA | |
| | ACCTGGACTGCCAGGAGGAGCCCGACAGCTG | |
| | CATCTCCGAGAAGCTGTTCATGGAGATGGCCG | |
| | AGCTGATGGTCTCCGAGGGCTGGAAGGACGC | |
| | CGGCTATGAGTACCTGTGCATTGATGACTGCT | |
| | GGATGGCCCCTCAAAGAGACTCTGAGGGCAG | |
| | ACTGCAGGCCGACCCCCAGAGATTCCCTCACG | |
| | GCATCAGACAGCTGGCCAACTACGTGCACAGC | |
| | AAGGGCCTGAAGCTGGGCATCTACGCCGATGT | |
| | GGGCAATAAGACCTGTGCCGGATTTCCTGGAT | |
| | CTTTTGGCTACTATGACATCGATGCCCAGACCT | |
| | TTGCCGACTGGGGAGTGGACCTGCTGAAGTTT | |
| | GATGGATGCTACTGTGACAGCCTGGAGAACCT | |
| | GGCCGACGGCTACAAGCACATGAGCCTGGCC | |
| | CTGAATAGAACAGGCAGAAGCATCGTGTACAG | |
| | CTGCGAGTGGCCCCTGTATATGTGGCCCTTCC | |
| | AAAAGCCTAACTACACCGAGATCAGGCAGTAC | |
| | TGCAACCACTGGAGAAACTTCGCCGACATCGA | |
| | TGACAGCTGGAAGAGCATCAAGAGCATCCTGG | |
| | ACTGGACAAGCTTCAACCAGGAGAGAATCGTG | |
| | GATGTGGCCGGCCCTGGCGGCTGGAATGACC | |
| | CTGACATGCTGGTCATTGGCAACTTCGGCCTG | |
| | AGCTGGAACCAGCAGGTGACACAGATGGCCCT | |
| | GTGGGCCATCATGGCCGCTCCCCTGTTCATGA | |
| | GCAACGACCTGAGACACATCAGCCCACAAGCC | |
| | AAGGCCCTGCTACAAGACAAGGACGTGATCGC | |
| | CATCAATCAAGACCCTCTGGGAAAGCAGGGCT | |
| | ACCAGCTGAGACAGGGCGACAACTTCGAAGTG | |
| | TGGGAGAGACCTCTGAGCGGACTGGCCTGGG | |
| | CCGTGGCCATGATCAACAGACAGGAGATCGGC | |
| | GGCCCCAGAAGCTACACCATCGCTGTGGCCTC | |
| | TCTGGGCAAGGGCGTGGCCTGCAACCCAGCC | |
| | TGCTTCATCACACAACTGCTGCCTGTGAAGAG | |
| | AAAGCTGGGCTTCTACGAGTGGACCAGCAGAC | |
| | TGAGAAGCCACATCAACCCCACCGGCACCGTG | |
| | CTGCTGCAACTGGAGAACACCATGCAGATGAG | |
| | CCTGAAGGACCTGCTGTGA | |
| α-GAL A | LDNGLARTPTMGWLHWERFMCNLDCQEEPDSC | 27 |
| | ISEKLFMEMAELMVSEGWKDAGYEYLCIDDCWM | |
| | APQRDSEGRLQADPQRFPHGIRQLANYVHSKGL | |
| | KLGIYADVGNKTCAGFPGSFGYYDIDAQTFADW | |
| | GVDLLKFDGCYCDSLENLADGYKHMSLALNRTG | |
| | RSIVYSCEWPLYMWPFQKPNYTEIRQYCNHWRN | |
| | FADIDDSWKSIKSILDWTSFNQERIVDVAGPGGW | |
| | NDPDMLVIGNFGLSWNQQVTQMALWAIMAAPLF | |
| | MSNDLRHISPQAKALLQDKDVIAINQDPLGKQGY | |
| | QLRQGDNFEVWERPLSGLAWAVAMINRQEIGGP | |
| | RSYTIAVASLGKGVACNPACFITQLLPVKRKLGFY | |
| | EWTSRLRSHINPTGTVLLQLENTMQMSLKDLL* | |

### Signal Peptide

Engineered peptides comprising α-GAL A may be further modified to increase efficiency, targeting, and stability of the peptide. Modifications may include alteration of signal peptide sequences, which in turn may be more effective in restoring α-GAL A function than wt amino acid sequences. Signal peptides are peptides that promote, targeting, localization, and provide other signals ques for a therapeutic peptide trafficking. A signal peptide (sp) may be a signal sequence, a targeting signal, a localization signal, a localization sequence, a transit peptide, a leader sequence, or a leader peptide. The engineered peptides may comprise an endogenous or an engineered sp. Sometimes, the engineered peptides may have more than one sp. Inclusion, deletion, alteration, substitution, or editing of a sp may alter protein function. Location of the sp peptide may also impact the activity. Additionally, sp sequences may be modified or synthesized to improve protein function, abundance, or activity. In the present technology, incorporation of different sp's into the engineered peptide sequence may enhance delivery, secretion, and/or overall efficacy of α-GAL A from the engineered peptide.

In some embodiments, the engineered peptide comprises a portion of signal peptide or a functional variant thereof. In some embodiments, the portion of the sp or the functional variant thereof is fused to or near the N terminus of the portion of α-GAL A or the functional variant thereof. In some aspects, the portion of the sp or the functional variant thereof is fused to or near the C terminus of the portion of α-GAL A or a functional variant thereof. In some aspects, the portion of the sp or functional variant thereof is fused internally within the portion of α-GAL A or a functional variant thereof.

In some embodiments, the engineered peptides comprise the endogenous human α-GAL A wt sp (SEQ ID NO: 28) or a functional variant thereof.

In some embodiments, the engineered peptide comprises a heterologous signal peptide or a functional variant thereof.

In some embodiments, the engineered peptide comprises the wt sp or a functional variant thereof, and a heterologous sp or a functional variant thereof. In some embodiments, the engineered peptide comprises two or more heterologous sp's, or their functional variants.

In certain aspects, the one or more heterologous signal peptides are selected from the group consisting of sp1, sp2, . . . sp22,the amino acid sequences of which are provided in Table 2. In certain embodiments, the one or more heterologous signal peptides are selected from the group consisting of sp1, sp3, sp4, sp18, sp20, sp21, and sp22.

In certain aspects, the wt sp or the functional variant thereof is encoded by a nucleotide sequence that is at least about 60%, 65%, 70%, 75%, 80%, 85%, 90%, 95%, 99%, or 100% identity to SEQ ID NO: 1 or 51. In certain aspects, the wt sp or the functional variant thereof is encoded by a nucleotide sequence that is at least 60%, 65%, 70%, 75%, 80%, 85%, 90%, 95%, 99%, or 100% identity to SEQ ID NO: 1 or 51. In certain aspects, the wt sp or the functional variant thereof is encoded by a nucleotide sequence that is about 60%, 65%, 70%, 75%, 80%, 85%, 90%, 95%, 99%, or 100% identity to SEQ ID NO: 1 or 51.

In certain embodiments, the one or more heterologous signal peptides or their functional variants are encoded by one or more nucleotide sequences that are at least about 60%, 65%, 70%, 75%, 80%, 85%, 90%, 95%, 99%, or 100% identity to one or more nucleotide sequences selected from the group consisting of SEQ ID NOs: 2, 3, 4, . . . , 23, 64, 65 and 66 as listed in Table 2. In certain aspects, the one or more heterologous signal peptides or their functional variants are encoded by one or more nucleotide sequences that are at least 60%, 65%, 70%, 75%, 80%, 85%, 90%, 95%, 99%, or 100% identity to one or more nucleotide sequences selected form the group consisting of SEQ ID NOs: 2, 3, 4, . . . , 23, 64, 65, and 66 as listed in Table 2. In certain aspects, the one or more heterologous signal peptides or their functional variants are encoded by one or more nucleotide sequences that are about 60%, 65%, 70%, 75%, 80%, 85%, 90%, 95%, 99%, or 100% identity to one or more nucleotide sequences selected from the group consisting of SEQ ID NOs: 2, 3, . . . , 23, 64, 65, and 66 as listed in Table 2.

TABLE 2
The endogenous human α-GAL A wt sp, embodiments of heterologous sp,
and embodiments of nucleotide sequences encoding the wt sp and heterologous sp.

| SEQUENCE | | |
|---|---|---|
| NAME | SEQUENCE | SEQ ID NO. |
| wt sp | MQLRNPELHLGCALALRFLALVSWDIPGARA | 28 |
| sp1 | MPSSVSWGILLLAGLCCLVPVSLA | 29 |
| sp2 | MNLLLILTFVAAAVA | 30 |
| sp3 | MAFLWLLSCWALLGTTFG | 31 |
| sp4 | MASRLTLLTLLLLLLAGDRASS | 32 |
| sp5 | MYRMQLLSCIALSLALVTNS | 33 |
| sp6 | MKWVTFISLLFLFSSAYS | 34 |
| sp7 | MLLLSALLLGLAHGYS | 35 |
| sp8 | MLLLSALLLGLAFGYS | 36 |
| sp9 | MLLLSALLLGLACGYS | 37 |
| sp10 | MLLSFALLLGLALGYS | 38 |
| sp11 | MLLVLALLLGLALGYS | 38 |
| sp12 | MLLLSALLLGLAMGYS | 40 |
| sp13 | MLLRIALLLGLAHGYS | 41 |
| sp14 | MLLLSALLLGLAIGYS | 42 |
| sp15 | MLLLPALLLGLAHGYS | 43 |
| sp16 | MLLVPALLLGLAHGYS | 44 |
| sp17 | MLLEHALLLGLAHGYS | 45 |
| sp18 | MGVKVLFALICIAVAEA | 46 |
| sp19 | MDIKVVFTLVFSALVQA | 47 |
| sp20 | MKIIILSVILAYCVTVNC | 48 |
| sp21 | MDAMKRGLCCVLLLCGAVFVSA | 49 |
| sp22 | MGVKVLFALICIAVAEVTG | 50 |
| wt SP | atgcagctgaggaacccagaactacatctgggctgcgcgcttgcgcttcgct | 1 |
| | tcctggccctcgtttcctgggacatccctggggctagagca | |
| Wt SP c.o. | ATGCAGCTGAGAAACCCCGAGCTGCACCTGGGCTG | 51 |
| | TGCCCTGGCCCTGCGCTTtCTGGCCCTgGTgAGTTG | |
| | GGAtATCCCtGGCGCCAGAGCC | |
| SP1 | atgccgtcttctgtctcgtggggcatcctcctgctggcaggcctgtgctgcctg | 2 |
| | gtccctgtctccctggct | |
| SP2 | atgaatctacttctgatccttacctttgttgcagctgctgttgct | 3 |
| SP3 | atggctttcctctggctcctctcctgctgggccctcctgggtaccaccttcggc | 4 |
| SP4 | atggcctccaggctgaccctgctgaccctcctgctgctgctgctggctgggg | 5 |
| | atagagcctcctca | |
| SP5 | atgtacaggatgcaactcctgtcttgcattgcactaagtcttgcacttgtcaca | 6 |
| | aacagt | |
| SP6 | atgaagtgggtaacctttatttcccttctttttctctttagctcggcttattcc | 7 |
| SP7 | atgctgctgttgtcagcgcttctccttgggcttgcccatgggtactcc | 8 |
| SP8 | atgctgctgttgtcagcgcttctccttgggcttgcctttgggtactcc | 9 |
| SP9 | atgctgctgttgtcagcgcttctccttgggcttgcctgtgggtactcc | 10 |
| SP10 | atgctgctgagtttcgcgcttctccttgggcttgcccttgggtactcc | 11 |
| SP11 | atgctgctggttttagcgcttctccttgggcttgcccttgggtactcc | 12 |
| SP12 | atgctgctgttgtcagcgcttctccttgggcttgccatggggtactcc | 13 |
| SP13 | atgctgctgagaatagcgcttctccttgggcttgcccatgggtactcc | 14 |
| SP14 | atgctgctgttgtcagcgcttctccttgggcttgccattgggtactcc | 15 |
| SP15 | atgctgctgttgccagcgcttctccttgggcttgcccatgggtactcc | 16 |
| SP16 | atgctgctggtgccagcgcttctccttgggcttgcccatgggtactcc | 17 |
| SP17 | atgctgctggagcacgcgcttctccttgggcttgcccatgggtactcc | 18 |
| SP18 | atgggagtgaaagttctttttgcccttatttgtattgctgtggccgaggcc | 19 |
| SP19 | atggatataaaggttgtctttactcttgttttctcagcattggttcaggca | 20 |
| SP20 | atgaagataataattctgtctgttatattggcctactgtgtcaccgtcaactgt | 21 |
| SP21 | atggatgcaatgaagagagggctctgctgtgtgctgctgctgtgtggagcag | 22 |
| | tcttcgtttcggca | |
| SP22 | atgggagtgaaagttctttttgcccttatttgtattgctgtggccgaggtgacgg | 23 |
| | ga | |
| Wt SP c.o.−1 | ATGCAACTGAGAAATCCTGAACTCCACCTGGGATGT | 64 |
| | GCCCTGGCTCTGAGATTTCTGGCCCTGGTGTCTTG | |
| | GGACATCCCTGGTGCTAGAGCC | |
| Wt SP c.o.−2 | ATGCAGCTGAGAAATCCTGAGCTGCACCTGGGCTG | 65 |
| | TGCCCTGGCTCTGAGATTTCTGGCTCTGGTGTCCT | |
| | GGGACATCCCTGGTGCTAGAGCC | |
| Wt SP c.o.−3 | ATGCAGCTGAGGAACCCAGAACTACATCTGGGCTG | 66 |
| | CGCGCTTGCGCTTCGCTTCCTGGCCCTCGTTTCCT | |
| | GGGACATCCCTGGGGCTAGAGCA | |

In some embodiments, the nucleotide sequence encoding the sp comprises or consists of SEQ ID NO: 1 or 51. In some aspects, the nucleotide sequence encoding the sp comprises or consists of a sequence that has at least about 60%, 65%, 70%, 75%, 80%, 85%, 90%, 95%, 99%, or 100% identity to SEQ ID NO: 1 or 51. In certain aspects, the nucleotide sequence encoding the sp comprises or consists of a sequence that has at least about 60%, 65%, 70%, 75%, 80%, 85%, 90%, 95%, 99%, or 100% identity to SEQ ID NO: 1 or 51. In certain aspects, the nucleotide sequence encoding the sp comprises or consists of a sequence that has about 60%, 65%, 70%, 75%, 80%, 85%, 90%, 95%, 99%, or 100% identity to SEQ ID NO: 1 or 51. In some embodiments, the sp is sp21. In some embodiments, the nucleotide sequence encoding the sp comprises or consists of SEQ ID NO: 22. In some aspects, the nucleotide sequence encoding the sp comprises or consists of a sequence that has at least about 60%, 65%, 70%, 75%, 80%, 85%, 90%, 95%, 99%, or 100% identity to SEQ ID NO: 22. In certain aspects, the nucleotide sequence encoding the sp comprises or consists of a sequence that has at least about 60%, 65%, 70%, 75%, 80%, 85%, 90%, 95%, 99%, or 100% identity to SEQ ID NO: 22. In certain aspects, the nucleotide sequence encoding the sp comprises or consists of a sequence that has about 60%, 65%, 70%, 75%, 80%, 85%, 90%, 95%, 99%, or 100% identity to SEQ ID NO: 22.

### Cell Penetrating Peptides

As stated previously, engineered peptides comprising α-GAL A may be further modified to increase efficiency, targeting, and stability of the peptide. Modifications may include inclusion or alteration of cell penetrating peptide sequences. Cell penetrating peptides may aid in the delivery of molecules and transport of peptides across cell or organelle membranes. Incorporation of a cell penetrating peptide sequence or a functional variant thereof into the engineered peptide sequence may promote delivery and/or secretion of the peptide. Location of the cell penetrating peptide within the peptide sequence may also impact the deliver and/or section of the peptide. Cell penetrating peptides may also be incorporated into engineered peptides. Described herein is the incorporation of TAT sequences into an engineered peptide. TAT sequences may comprise arginine-rich sequences which directly penetrate cell or organelle membranes. In the present technology, incorporation of TAT or other cell penetrating peptides into the engineered peptide sequence may enhance delivery, secretion, and/or overall efficacy of α-GAL A from the engineered peptide. Examples of cell penetrating peptides include, without limitation, TAT p47-57, TAT p48-60, Penetratin p43-58, hCT p-9-32, polyarginies, pVEC, Pep-1, transportan, and MAP.

Some aspects of the present technology describe the use of an engineered peptide comprising α-GAL A and a cell penetrating peptide, which may be incorporated into the engineered peptide sequence to modulate different peptide characteristics including, but not limited to, enhanced peptide secretion and delivery to target cells or tissues. In some embodiments, the engineered peptide comprises a cell penetrating peptide or a functional variant thereof. In some embodiments, the cell penetrating peptide comprises or consists of at least a portion of TAT p47-57 (SEQ ID NO: 55) or a functional variant thereof. In certain aspects, the cell penetrating peptide is encoded by a nucleotide sequence that comprises at least about 60-100% identity to SEQ ID NO: 24. In certain aspects, the cell penetrating peptide is encoded by a nucleotide sequence that comprises at least about 60%, 65%, 70%, 75%, 80%, 85%, 90%, 95%, 99%, or 100% identity to SEQ ID NO: 24.

In certain aspects, the cell penetrating peptide is encoded by a nucleotide sequence that comprises at least 60-100% identity to SEQ ID NO: 24. In certain aspects, the cell penetrating peptide is encoded by a nucleotide sequence that comprises at least 60%, 65%, 70%, 75%, 80%, 85%, 90%, 95%, 99%, or 100% identity to SEQ ID NO: 24.

In certain aspects, the cell penetrating peptide is encoded by a nucleotide sequence that comprises about 60-100% identity to SEQ ID NO: 24. In certain aspects, the cell penetrating peptide is encoded by a nucleotide sequence that comprises about 80%, 60%, 65%, 70%, 75%, 85%, 90%, 95%, 99%, or 100% identity to SEQ ID NO: 24.

In some embodiments, the cell penetrating peptide comprises or consists of at least a portion of a peptide selected from the group consisting of TAT p47-57 (SEQ ID NO: 55), TAT p48-60 (SEQ ID NO: 56), Penetratin p43-58 (SEQ ID NO: 57), hCT p9-32 (SEQ ID NO: 58), polyarginines (SEQ ID NO: 59), pVEC (SEQ ID NO: 60), Pep-1 (SEQ ID NO: 61), Transportan (SEQ ID NO: 62), and MAP (SEQ ID NO: 63), or a functional variant thereof. In certain aspects, the cell penetrating peptide is encoded by a nucleotide sequence that comprises at least about 60-100% identity to a nucleotide sequence selected from the group consisting of SEQ ID NOs: 24, 52, 53, and 54. In certain aspects, the cell penetrating peptide is encoded by a nucleotide sequence that comprises at least about 60%, 65%, 70%, 75%, 80%, 85%, 90%, 95%, 99%, or 100% identity to a nucleotide sequence selected from the group consisting of SEQ ID NOs: 24, 52, 53, and 54.

In certain aspects, the cell penetrating peptide is encoded by a nucleotide sequence that comprises at least 60-100% identity to a nucleotide sequence selected from the group consisting of SEQ ID NOs: 24, 52, 53, and 54. In certain aspects, the cell penetrating peptide is encoded by a nucleotide sequence that comprises at least 60%, 65%, 70%, 75%, 80%, 85%, 90%, 95%, 99%, or 100% identity to a nucleotide sequence selected from the group consisting of SEQ ID NOs: 24, 52, 53, and 54.

In certain aspects, the cell penetrating peptide is encoded by a nucleotide sequence that comprises about 60-100% identity to a nucleotide sequence selected from the group consisting of SEQ ID NOs: 24, 52, 53, and 54. In certain aspects, the cell penetrating peptide is encoded by a nucleotide sequence that comprises about 80%, 60%, 65%, 70%, 75%, 85%, 90%, 95%, 99%, or 100% identity to a nucleotide sequence selected from the group consisting of SEQ ID NOs: 24, 52, 53, and 54.

In some embodiments, the cell penetrating peptide is fused to the C terminus of the α-GAL A. In some aspects, the cell penetrating peptide is fused to the N terminus of the α-GAL A. In certain aspects, the cell penetrating peptide is fused internally within the α-GAL A.

In certain aspects, the engineered peptide comprises more than one cell penetrating peptide or functional variant thereof. In some aspects, the engineered peptide comprises a first cell penetrating peptide or functional variant thereof fused to or near the N terminus of the α-GAL A, and a second cell penetrating peptide or functional variant thereof fused to or near the C terminus of the α-GAL A. In some aspects, the engineered peptide comprises a first cell penetrating peptide or functional variant thereof fused to or near the N terminus of the α-GAL A, and a second cell penetrating or functional variant thereof fused internally within the α-GAL A. In some aspects, the engineered peptide comprises a first cell penetrating peptide or functional variant thereof fused to or near the C terminus of the α-GAL A, and a second cell penetrating peptide or functional variant thereof fused internally within the α-GAL A.

In some aspects, the engineered peptide comprises a first cell penetrating peptide or functional variant thereof fused to or near the N terminus of the α-GAL A, a second cell penetrating peptide or functional variant thereof fused to or near the C terminus of the α-GAL A, and a third cell penetrating peptide or functional variant thereof fused internally within the α-GAL A.

TABLE 3
Examples of cell-penetrating peptide and nucleotide sequences encoding
cell-penetrating peptide.

| SEQUENCE | | SEQ ID |
|---|---|---|
| NAME | SEQUENCE | NO. |
| TAT p47-57 | TATGGCAGGAAGAAGCGGAGACAGCGACGAAGA | 24 |
| TAT p48-60 | GGCAGGAAGAAGCGGAGACAGCGACGAAGACCTCCTCAA | 52 |
| Penetratin | CGCCAGATAAAGATTTGGTTCCAGAATCGGCGCATGAAGTG | 53 |
| p43-58 | GAAGAAG | |
| hCT p9-32 | CTGGGCACATACACGCAGGACTTCAACAAGTTTCACACGTT | 54 |
| | CCCCCAAACTGCAATTGGGGTTGGAGCACCT | |
| Tat p47-57 | YGRKKRRQRRR | 55 |
| TAT p48-60 | GRKKRRQRRRPPQ | 56 |
| Penetratin | RQIKIWFQNRRMKWKK | 57 |
| p43-58 | | |
| hCT p9-32 | LGTYTQDFNKFHTFPQTAIGVGAP | 58 |
| Polyarginies | RRRRRRRRR | 59 |
| pVEC | LLIILRRRIRKQAHAHSK | 60 |
| Pep-1 | KETWWETWWTEWSQPKKKRKV | 61 |
| Transportan | GWTLNSAGYLLGKINLKALAALAKKIL | 62 |
| MAP | KLALKLALKALKAALKLA | 63 |

### Viral Vectors

Therapies which promote increased α-GAL A activity are sought out for Fabry disease treatment. One potential therapy is delivering α-GAL A to a subject via engineered peptides which are expressed from viral vectors. Viral vectors may be constructed to deliver genetic material and express engineered peptides within a cell or tissue of a subject in need thereof. The viral genome can be modified for the inclusion or exclusion of genetic elements, one being an expression cassette which comprises the nucleic acid sequence to be expressed as protein. The viral vector may be designed or synthesized in vivo or in vitro, and may be a retrovirus, lentivirus, poxvirus, adenovirus, and adeno-associated virus. Aspects of the present technology include an rAAV vector comprising an rAAV genome, wherein the rAAV genome comprises a nucleic acid sequence that encodes the engineered peptide comprising α-GAL A. The viral vector may also be single-stranded or self-complimentary, referred to as an ss-rAAV or an sc-rAAV, respectively. Furthermore, the viral vector may be modified through encapsidation or serotype selection, each of which modifies efficacy of the viral vector. Described herein is the use of vectors to increase expression and delivery of α-GAL A.

In some embodiments, the viral vector is encapsidated. In some aspects, the viral vector comprises a serotype. In certain aspects, the serotype is rAAV1, rAAV2, rAAV3, rAAV4, rAAV5, rAAV6, rAAV7, rAAV8, rAAV9, rAAV10, rAAV11, rAAV12, or rAAV13. In certain aspects, the serotype may be derivatives of rAAV1, rAAV2, rAAV3, rAAV4, rAAV5, rAAV6, rAAV7, rAAV8, rAAV9, rAAV10, rAAV11, rAAV12, or rAAV13.

In some embodiments, the viral vector further comprises a promoter. In some aspects, the viral vector is a tissue-specific promoter. In some aspects, the viral vector is a liver, heart, kidney, or spleen-specific promoter. In certain aspects, the liver-specific promoter is TTR, HBV, ALB, human α-1 antitrypsin (hAAT) promoter or phosphoenolpyruvate carboxykinase (PEPCK) gene promoter. In certain aspects, the liver-specific promoter is engineered liver-specific promoter.

In certain aspects, the viral vector comprises at least one ITR nucleotide sequence. In certain aspects, the viral vector comprises a 5′ITR and a 3′ITR. In certain aspects, the viral vector does not comprise any intron. In certain aspects, the viral vector comprises at least one intron. In certain aspects, the vector comprises a polyadenylation (polyA) nucleotide sequence. In certain aspects, the viral vector comprises at least one ITR nucleotide sequence, an intron, and/or a polyA nucleotide sequence. Examples of ss-rAAV and sc-rAAV are shown in FIGS. 1A and 1B.

In some aspects, the viral vector comprises a 5′ITR, a 3′ITR, a tissue-specific promoter, an intron, a stop codon, a polyA nucleotide sequence, an hGLA nucleotide sequence, and a nucleotide sequence encoding an sp. In certain embodiments, the hGLA nucleotide sequence is wt hGLA (SEQ ID NO: 25) or hGLA c.o.v1 (SEQ ID NO: 26). In certain aspects, the sp is a wt sp or a sp21. In certain aspects, the sp nucleotide sequence is a sp nucleotide sequence listed in Table 2. In certain aspects, the tissue-specific promoter is TTR. In certain aspects, the viral vector is an ss-rAAV vector or an sc-rAAV vector.

In some embodiments, the viral vector is rAAV8. In some aspects, the sc-rAAV vector is ss-wtspGLA or ss-sp21 GLA. In some aspects, the ss-rAAV vector is FD802-1, FD802-2, FD802-3, FD802-4, or FD802-5.

In some aspects, the hGLA sequence is expressed from a vector. In certain aspects, a vector comprising hGLA is ss-rAAV expressing α-GAL A+wt sp, ss-rAAV expressingα-GAL A+sp21, FD802-1, or FD802-2. In certain aspects, a vector comprising hGLA c.o.v1 (SEQ ID NO: 26) is FD802-3, FD802-4, or FD802-5.

In some aspects, the signal peptide is expressed from a vector. In some aspects, the vector expressing a wt sp peptide is ss-rAAV expressing α-GAL A+wt sp, FD802-1, FD802-3, or FD802-5. In some aspects, the vector expressing a sp21 peptide is ss-rAAV expressing α-GAL A+sp21, FD802-2, or FD802-4.

In some aspects, the cell penetrating peptide is expressed from a vector. In some aspects, the vector expressing a cell penetrating peptide is FD802-5.

### Pharmaceutical Compositions

The engineered peptides and viral vectors described herein may be formulated in a pharmaceutical composition comprising, consisting essentially of, or consisting of an effective amount of one or more engineered peptides or viral vectors described herein. Pharmaceutical compositions may be modified for different routes of administration or to increase composition stability. Additionally, pharmaceutical compositions may be modified to deliver different dosages for the active ingredient, such as an engineered peptide, a viral vector, and/or a viral composition. In some embodiments, the pharmaceutical composition further comprises one or more additional therapeutic agents, which are not the engineered peptides or viral vectors disclosed herein. In some embodiments, the pharmaceutical composition further comprises a pharmaceutically acceptable carrier, excipient, additive, preservative, or a combination thereof. Examples of acceptable carriers include physiologically acceptable solutions, such as sterile saline and sterile buffered saline.

In some embodiments, the engineered peptide is present in a pharmaceutical composition. In some embodiments, the viral vector is present in a pharmaceutical composition. In some aspects, the pharmaceutical composition comprises an encapsidated vector.

In some aspects, the pharmaceutical composition further comprises an excipient. In some aspects, the pharmaceutical composition further comprises a pharmaceutically-acceptable carrier. In some aspects, the pharmaceutical composition further comprises a pharmaceutically acceptable carrier and/or excipient. In some embodiments, the pharmaceutically acceptable carrier and/or excipient comprises water.

In some aspects, the pharmaceutical composition is formulated for intraperitoneal, intravenous, parenteral, subcutaneous, intramuscular, intracerebroventricular, or oral administration. In some embodiments, the single pharmaceutical composition is formulated for subcutaneous administration.

In some aspects, the pharmaceutical composition is administered with a second Fabry disease therapeutic. In certain aspects, the second Fabry disease therapeutic comprises an engineered peptide, vector, or viral vector.

Viral vectors may be formulated into a pharmaceutical composition, or viral composition, to deliver genetic material to a subject in need thereof to express a target or therapeutic peptide. In some embodiments, the present technology provides a viral composition for increasing α-GAL A expression in a subject in need thereof. In other embodiments, the present technology provides a viral composition for treating Fabry disease or alleviating symptoms of Fabry disease in a subject in need thereof. The α-GAL A of the viral vector is expressed from an expression cassette, further comprising at least one ITR nucleotide sequence, an intron, and/or a polyadenylation polyA nucleotide sequence. Viral composition may be modified such that expression of the peptide of interest, such as those comprising α-GAL A, is increased. Other parameters of viral compositions may be modified to increase composition stability or delivery efficiency to a target cell or tissue. This includes, but is not limited to, encapsidation of viral vector which may enhance delivery and stability, and serotype selection, which may permit binding to specific cell surface proteins of interest.

In some aspects, the viral composition comprises a viral vector expressing α-GAL A. In some aspects, the viral vector is encapsidated. In some aspects, the viral vector is an rAAV vector. In certain aspects, the viral vector is an sc-rAAV or ss-rAAV vector. In certain aspects, the viral vector comprises the serotype rAAV1, rAAV2, rAAV3, rAAV4, rAAV5, rAAV6, rAAV7, rAAV8, rAAV9, rAAV10, rAAV11, rAAV12, or rAAV13.

In some embodiments, the viral composition is formulated for intraperitoneal, intravenous, parenteral, subcutaneous, intramuscular, intracerebroventricular, or oral administration. In some embodiments, the single pharmaceutical composition is formulated for subcutaneous administration.

### Associated Methods

The engineered peptides, viral vectors, and pharmaceutical compositions described herein are intended for use in a method of treating Fabry disease or reducing symptoms associated with Fabry disease. Symptoms associated with Fabry disease include, but are not limited to, angiokeratomas, acroparesthesia, hypohidrosis/anhidrosis, corneal, lenticular opacity, cardiac problems, pain, a reduction in kidney function, or any symptom resulting from α-GAL A dysfunction or deficiency.

In some embodiments, the present technology provides a method for treating Fabry disease in a subject. In some embodiments, the present technology provides a method for reducing one or more symptoms associated with Fabry disease in a subject. In some embodiments, the present technology provides a method for increasing α-GAL A expression in a subject. In some aspects, the α-GAL expression and/or activity is increased in a target tissue and/or plasma of a subject. In certain aspects, the target tissue is liver, heart, kidney, and/or spleen.

In some embodiments, the present technology provides for a method of treating Fabry disease or reducing one or more symptoms of Fabry disease in a subject, comprising administering the engineered peptide comprising α-GAL A, the viral vector expressing α-GAL A, or a pharmaceutical composition comprising the engineered peptide comprising α-GAL A or the viral vector expressing α-GAL A to the subject in need thereof.

In some embodiments, the present technology provides for a method of increasing α-Gal A expression in a subject, comprising administering the engineered peptide comprising α-GAL A, the viral vector expressing α-GAL A, or a pharmaceutical composition comprising the engineered peptide comprising α-GAL A or the viral vector expressing α-GAL A to the subject in need thereof.

In some embodiments, the present technology provides a method for reducing lyso-Gb3 in a target tissue and/or plasma of the subject. In some aspects, the present technology provides a method for increasing glycosylation of α-GAL A in a target tissue and/or plasma of the subject. In some aspects, the present technology provides a method for increasing mature α-GAL A in a target tissue and/or plasma of the subject.

In some aspects, the present technology provides a method for modulating sphingolipid storage in a target tissue and/or plasma of the subject. In certain aspects, the modulation of sphingolipid storage is reducing or increasing sphingolipids in a target tissue of a target tissue and/or plasma of the subject. In some embodiments, the present technology provides for a method of reducing sphingolipid storage in one or more target tissues of a subject, comprising administering the engineered peptide comprising α-GAL A, the viral vector expressing α-GAL A, or a pharmaceutical composition comprising the engineered peptide comprising α-GAL A or the viral vector expressing α-GAL A to the subject in need thereof.

### Dosages

Depending on the indication, severity, and administration route, a suitable dose may be selected accordingly. For example, for acute indications, fewer treatments with a higher dose in each treatment are administered; while for chronic indications requiring frequent and long-term treatment, a lower dose treatment is administered. In tissues with high lyso-Gb3 levels or low α-GAL A expression, a high dose of engineered peptide, viral vector/and or pharmaceutical composition may be required. Dosage may be determined by the effective amount of engineered peptide, viral vector, and/or pharmaceutical composition to improve symptoms or molecular outcomes associated with Fabry disease and reduced α-GAL A expression.

The dosage of engineered peptide, viral vector, and or pharmaceutical composition which express α-GAL A expression may be determined by the effective dose which increases α-GAL A expression and/or activity, decreases lyso-GB3, or decreases symptoms associated with Fabry disease. The engineered peptide, viral vector/and or pharmaceutical composition may be administered to the subject in need thereof in a single dose or multiple doses.

In some embodiments, the engineered peptide, viral vector, and or pharmaceutical composition is administered to the subject for once.

In some aspects, the engineered peptide, viral vector, and or pharmaceutical composition is administered with a second Fabry disease treatment.

It is to be understood that, for the subject, specific dosage regimes should be adjusted over time according to the severity of disease or symptoms associated with disease of the subject in need thereof. For example, the dosage of the engineered peptide, viral vector/and or pharmaceutical composition may be increased if the lower dose does not provide sufficient therapeutic activity.

In some embodiments, dosage is determined by the effective amount of engineered peptide, viral vector, and/or pharmaceutical composition to reduce one or more symptoms associated with Fabry disease. In some embodiments, dosage is determined by the effective amount of engineered peptide, viral vector, and/or pharmaceutical composition to increase α-GAL A expression and/or activity. In some embodiments, dosage is determined by the effective amount of engineered peptide, viral vector, and/or pharmaceutical composition to reduce plasma and/or tissue lyso-Gb3 levels.

Dosage of viral vectors and/or pharmaceutical compositions comprising viral vectors may be adjusted according to specific load of viral genome desired to be delivered to the subject in need thereof.

In some embodiments, the engineered peptide, viral vector, and/or pharmaceutical composition is administered at a dose of at least about 0.5E+10 vg/kg to 14E+14 vg/kg.

In some embodiments, the engineered peptide, viral vector, and/or pharmaceutical composition is administered at a dose of at least about 0.5E+10 vg/kg to 7E+10 vg/kg. In some embodiments, the engineered peptide, viral vector, and/or pharmaceutical composition is administered at a dose of at least about 0.5E+10 vg/kg, at least about 1 E+10 vg/kg, at least about 1.5E+10 vg/kg, at least about 2E+10 vg/kg, at least about 2.5E+10 vg/kg, at least about 3E+10 vg/kg, at least about 3.5E+10 vg/kg, at least about 4E+10 vg/kg, at least about 4.5E+10 vg/kg, at least about 5E+10 vg/kg, at least about 5.5E+10 vg/kg, at least about 6E+10 vg/kg, at least about 6.5E+10 vg/kg, or at least about 7E+10 vg/kg.

In some embodiments, the engineered peptide, viral vector, and/or pharmaceutical composition is administered at a dose of at least 0.5E+10 vg/kg to 7E+10 vg/kg. In some aspects, the engineered peptide, viral vector, and/or pharmaceutical composition is administered at a dose of at least 0.5E+10 vg/kg, at least 1 E+10 vg/kg, at least 1.5E+10 vg/kg, at least 2E+10 vg/kg, at least 2.5E+10 vg/kg, at least 3E+10 vg/kg, at least 3.5E+10 vg/kg, at least about 4E+10 vg/kg, at least 4.5E+10 vg/kg, at least 5E+10 vg/kg, at least 5.5E+10 vg/kg, at least 6E+10 vg/kg, at least 6.5E+10 vg/kg, or at least 7E+10 vg/kg.

In some embodiments, the engineered peptide, viral vector, and/or pharmaceutical composition is administered at a dose of about 0.5E+10 vg/kg to 7E+10 vg/kg. In some aspects, the engineered peptide, viral vector, and/or pharmaceutical composition is administered at a dose of about 0.5E+10 vg/kg, about 1 E+10 vg/kg, about 1.5E+10 vg/kg, about 2E+10 vg/kg, about 2.5E+10 vg/kg, about 3E+10 vg/kg, about 3.5E+10 vg/kg, about 4E+10 vg/kg, about 4.5E+10 vg/kg, about 5E+10 vg/kg, about 5.5E+10 vg/kg, about 6E+10 vg/kg, about 6.5E+10 vg/kg, about 7E+10 vg/kg.

In some embodiments, the engineered peptide, viral vector, and/or pharmaceutical composition is administered at a dose of at least about 0.5E+11 vg/kg to 7E+11 vg/kg. In some embodiments, the engineered peptide, viral vector, and/or pharmaceutical composition is administered at a dose of at least about 0.5E+11 vg/kg, at least about 1 E+11 vg/kg, at least about 1.5E+11 vg/kg, at least about 2E+11 vg/kg, at least about 2.5E+11 vg/kg, at least about 3E+11 vg/kg, at least about 3.5E+11 vg/kg, at least about 4E+11 vg/kg, at least about 4.5E+11 vg/kg, at least about 5E+11 vg/kg, at least about 5.5E+11 vg/kg, at least about 6E+11 vg/kg, at least about 6.5E+11 vg/kg, or at least about 7E+11 vg/kg.

In some embodiments, the engineered peptide, viral vector, and/or pharmaceutical composition is administered at a dose of at least 0.5E+11 vg/kg to 7E+11 vg/kg. In some aspects, the engineered peptide, viral vector, and/or pharmaceutical composition is administered at a dose of at least 0.5E+11 vg/kg, at least 1 E+11 vg/kg, at least 1.5E+11 vg/kg, at least 2E+11 vg/kg, at least 2.5E+11 vg/kg, at least 3E+11 vg/kg, at least 3.5E+11 vg/kg, at least about 4E+11 vg/kg, at least 4.5E+11 vg/kg, at least 5E+11 vg/kg, at least 5.5E+11 vg/kg, at least 6E+11 vg/kg, at least 6.5E+11 vg/kg, or at least 7E+11 vg/kg.

In some embodiments, the engineered peptide, viral vector, and/or pharmaceutical composition is administered at a dose of about 0.5E+11 vg/kg to 7E+11 vg/kg. In some aspects, the engineered peptide, viral vector, and/or pharmaceutical composition is administered at a dose of about 0.5E+11 vg/kg, about 1 E+11 vg/kg, about 1.5E+11 vg/kg, about 2E+11 vg/kg, about 2.5E+11 vg/kg, about 3E+11 vg/kg, about 3.5E+11 vg/kg, about 4E+11 vg/kg, about 4.5E+11 vg/kg, about 5E+11 vg/kg, about 5.5E+11 vg/kg, about 6E+11 vg/kg, about 6.5E+11 vg/kg, about 7E+11 vg/kg.

In some embodiments, the engineered peptide, viral vector, and/or pharmaceutical composition is administered at a dose of at least about 0.5E+12 vg/kg to 7E+12 vg/kg. In some embodiments, the engineered peptide, viral vector, and/or pharmaceutical composition is administered at a dose of at least about 0.5E+12 vg/kg, at least about 1 E+12 vg/kg, at least about 1.5E+12 vg/kg, at least about 2E+12 vg/kg, at least about 2.5E+12 vg/kg, at least about 3E+12 vg/kg, at least about 3.5E+12 vg/kg, at least about 4E+12 vg/kg, at least about 4.5E+12 vg/kg, at least about 5E+12 vg/kg, at least about 5.5E+12vg/kg, at least about 6E+12 vg/kg, at least about 6.5E+12 vg/kg, or at least about 7E+12 vg/kg.

In some embodiments, the engineered peptide, viral vector, and/or pharmaceutical composition is administered at a dose of at least 0.5E+12 vg/kg to 7E+12 vg/kg. In some embodiments, the engineered peptide, viral vector, and/or pharmaceutical composition is administered at a dose of at least 0.5E+12 vg/kg, at least 1 E+12 vg/kg, at least 1.5E+12 vg/kg, at least 2E+12 vg/kg, at least 2.5E+12 vg/kg, at least 3E+12 vg/kg, at least 3.5E+12 vg/kg, at least 4E+12 vg/kg, at least 4.5E+12 vg/kg, at least 5E+12 vg/kg, at least 5.5E+12 vg/kg, at least 6E+12 vg/kg, at least 6.5E+12 vg/kg, or at least 7E+12 vg/kg.

In some embodiments, the engineered peptide, viral vector, and/or pharmaceutical composition is administered at a dose of about 0.5E+12 vg/kg to 7E+12 vg/kg. In some embodiments, the engineered peptide, viral vector, and/or pharmaceutical composition is administered at a dose of about 0.5E+12 vg/kg, about 1 E+12 vg/kg, about 1.5E+12 vg/kg, about 2E+12 vg/kg, about 2.5E+12 vg/kg, about 3E+12 vg/kg, about 3.5E+12 vg/kg, about 4E+12 vg/kg, about 4.5E+12 vg/kg, about 5E+12 vg/kg, about 5.5E+12 vg/kg, about 6E+12 vg/kg, about 6.5E+12 vg/kg, about 7E+12 vg/kg.

In some embodiments, the engineered peptide, viral vector, and/or pharmaceutical composition is administered at a dose of at least about 0.5E+13 vg/kg to 7E+13 vg/kg. In some embodiments, the engineered peptide, viral vector, and/or pharmaceutical composition is administered at a dose of at least about 0.5E+13 vg/kg, at least about 1 E+13 vg/kg, at least about 1.5E+13 vg/kg, at least about 2E+13 vg/kg, at least about 2.5E+13 vg/kg, at least about 3E+13 vg/kg, at least about 3.5E+13 vg/kg, at least about 4E+13 vg/kg, at least about 4.5E+13 vg/kg, at least about 5E+13 vg/kg, at least about 5.5E+13 vg/kg, at least about 6E+13 vg/kg, at least about 6.5E+13 vg/kg, or at least about 7E+13 vg/kg.

In some embodiments, the engineered peptide, viral vector, and/or pharmaceutical composition is administered at a dose of at least 0.5E+13 vg/kg to 7E+13 vg/kg. In some embodiments, the engineered peptide, viral vector, and/or pharmaceutical composition is administered at a dose of at least 0.5E+13 vg/kg, at least 1 E+13 vg/kg, at least 1.5E+13 vg/kg, at least 2E+13 vg/kg, at least 2.5E+13 vg/kg, at least 3E+13 vg/kg, at least 3.5E+13 vg/kg, at least 4E+13 vg/kg, at least 4.5E+13 vg/kg, at least 5E+13 vg/kg, at least 5.5E+13 vg/kg, at least 6E+13 vg/kg, at least 6.5E+13 vg/kg, or at least 7E+13 vg/kg.

In some embodiments, the engineered peptide, viral vector, and/or pharmaceutical composition is administered at a dose of about 0.5E+13 vg/kg to 7E+13 vg/kg. In some embodiments, the engineered peptide, viral vector, and/or pharmaceutical composition is administered at a dose of about 0.5E+13 vg/kg, about 1 E+13 vg/kg, about 1.5E+13 vg/kg, about 2E+13 vg/kg, about 2.5E+13 vg/kg, about 3E+13 vg/kg, about 3.5E+13 vg/kg, about 4E+13 vg/kg, about 4.5E+13 vg/kg, about 5E+13 vg/kg, about 5.5E+13 vg/kg, about 6E+13 vg/kg, about 6.5E+13 vg/kg, about 7E+13 vg/kg.

In some embodiments, the engineered peptide, viral vector, and/or pharmaceutical composition is administered at a dose of at least about 0.5E+14 vg/kg to 7E+14 vg/kg. In some embodiments, the engineered peptide, viral vector, and/or pharmaceutical composition is administered at a dose of at least about 0.5E+14 vg/kg, at least about 1 E+14 vg/kg, at least about 1.5E+14 vg/kg, at least about 2E+14 vg/kg, at least about 2.5E+14 vg/kg, at least about 3E+14 vg/kg, at least about 3.5E+14 vg/kg, at least about 4E+14 vg/kg, at least about 4.5E+14 vg/kg, at least about 5E+14 vg/kg, at least about 5.5E+14 vg/kg, at least about 6E+14 vg/kg, at least about 6.5E+14 vg/kg, or at least about 7E+14 vg/kg.

In some embodiments, the engineered peptide, viral vector, and/or pharmaceutical composition is administered at a dose of at least 0.5E+14 vg/kg to 7E+14 vg/kg. In some aspects, the engineered peptide, viral vector, and/or pharmaceutical composition is administered at a dose of at least 0.5E+14 vg/kg, at least 1 E+14 vg/kg, at least 1.5E+14 vg/kg, at least 2E+14 vg/kg, at least 2.5E+14 vg/kg, at least 3E+14 vg/kg, at least 3.5E+14 vg/kg, at least 4E+14 vg/kg, at least 4.5E+14 vg/kg, at least 5E+14 vg/kg, at least 5.5E+14 vg/kg, at least 6E+14 vg/kg, at least 6.5E+14 vg/kg, or at least 7E+14 vg/kg.

In some embodiments, the engineered peptide, viral vector, and/or pharmaceutical composition is administered at a dose of about 0.5E+14 vg/kg to 7E+14 vg/kg. In some aspects the engineered peptide, viral vector, and/or pharmaceutical composition is administered at a dose of about 0.5E+14 vg/kg, about 1 E+14 vg/kg, about 1.5E+14 vg/kg, about 2E+14 vg/kg, about 2.5E+14 vg/kg, about 3E+14 vg/kg, about 3.5E+14 vg/kg, about 4E+14 vg/kg, about 4.5E+14 vg/kg, about 5E+14 vg/kg, about 5.5E+14 vg/kg, about 6E+14 vg/kg, about 6.5E+14 vg/kg, about 7E+14 vg/kg.

It will be apparent to one skilled in the art that various equivalents, changes, and modifications may be made without departing from the scope of present technology, and it is understood that such equivalent embodiments, are to be included herein.

The following examples are intended to illustrate various embodiments of the present technology. As such, the specific embodiments discussed are not to be constructed as limitations on the scope of the present technology.

### Examples

Reduced α-galactosidase A (α-GAL A) activity or expression may lead to Fabry disease.

α-GAL A function may be restored using gene therapy approaches, such as, by exogenous expression of α-GAL A using a viral vector. To determine if vector-mediated delivery of α-GAL A could be achieved, a series of viral vectors engineered to express α-GAL A were synthesized (FIGS. 1A and 1B). Each vector comprised an expression cassette having either a wt hGLA nucleotide sequence (SEQ ID NO: 25), or functional variant thereof, such as hGLA c.o.v1 (SEQ ID NO: 26), and the endogenous wt signal peptide (wt sp) nucleotide sequence of hGLA or a heterologous sp nucleotide sequence, such as sp1-sp22 shown in Table 2.

Vector generation: mRNA was extracted from hEK293 cells and reverse-transcribed into cDNA. The wt signal peptide of hGLA was substituted with a heterologous sp sequence at the N-terminus by cloning PCR fragments into a restriction digested plasmid. Nucleotide sequences encoding α-GAL A fused with wt sp or any one of sp1 to sp22 were cloned into expression constructs containing a liver specific TTR promoter and a polyadenylation sequence flanked by AAV2 inverted terminal repeat (ITR). Vectors were either constructed using a single-stranded rAAV8 vector (ss-rAAV; FIG. 1A) or constructed using a self-complimentary rAAV8 vector (sc-rAAV; FIG. 1B), and sequences were verified by enzyme digestion and Sanger sequencing.

Unless specified otherwise, in the Example section, low dose refers to 2E+12 vg/kg, medium dose refers to 5E+12 vg/kg, high dose refers to 5E+13 vg/kg, wild type mice refers to healthy mice who did not have Fabry disease and were treated with PBS, untreated refers to Fabry mice treated with PBS, which is the same vehicle used for the vector injections without vector, Rec.hAGA refers to recombinant human α-GAL A which was used as positive control for in vitro glycosylation test.

### Example 1: rAAV's Expressing α-GAL A Fused with Several Heterologous Sp's Increased α-GAL A Expression and Activity in HepG2 Cells and Fabry Mice

To determine whether incorporation of heterologous sp into α-GAL A increases α-GAL A expression, HepG2 cells were transfected with plasmids expressing α-GAL A fused to one sp selected from the group consisting of sp1, sp2, . . . , and sp22. α-GAL A was detected in culture media using western blot (WB) techniques. α-GAL A fused to sp1, sp3, sp4, sp18, and sp20-22 correlated with increased extracellular secretion of α-GAL A (FIG. 2A) and α-GAL A activities (FIG. 2B) compared to α-GAL A fused to wt sp. α-GAL A fused to heterologous sp demonstrated increased activity in HepG2 culture media relative to α-GAL A fused to wt sp. This suggests that α-GAL A expression and secretion may be increased by substituting wt sp for a heterologous sp.

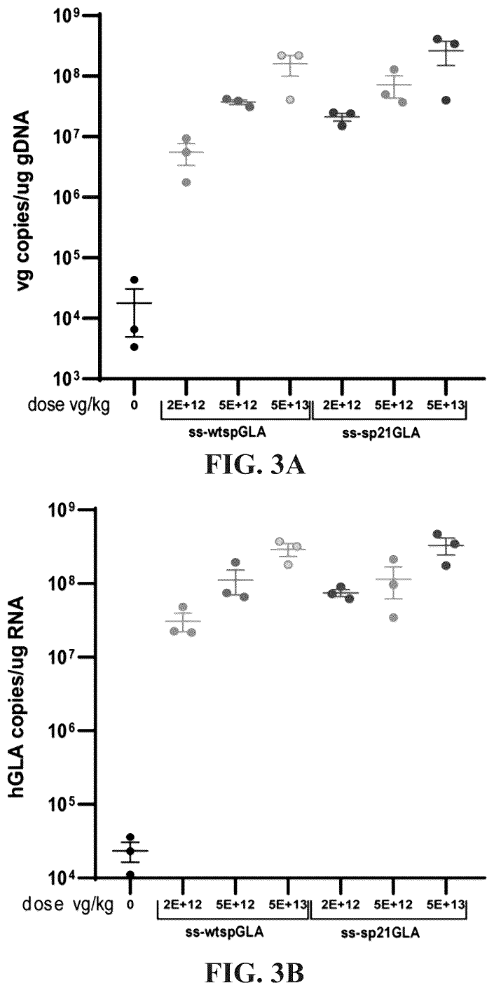

To assess genome expression and distribution of ss-sp21GLA following injection, rAAV genomic DNA (gDNA) and hGLA RNA were measured in Fabry mice liver 12-weeks following injection with either low dose, medium dose, or high dose ss-wtspGLA or ss-sp21 GLA. rAAV8 genome distribution in liver quantified at 12 weeks post injection showed a dose-dependent effect, with higher levels observed at the high dose with both vectors, demonstrating higher transduction efficiencies than those injected with the corresponding lower dose of each vector (FIG. 3A). mRNA levels of engineered hGLA in the liver were also analyzed at 12 weeks post injection, and the same trends as that of the rAAV8 genome were observed (FIG. 3B). AAV vectors were transduced in mice liver as the AAV genomes were detected from the liver genome samples, in both vector-treated groups. The transduced AAV vectors were transcribed as the transgene mRNA was detected from the liver mRNA samples in both vector-treated groups. The successful AAV transduction and transcription is the premise for effective treatment.

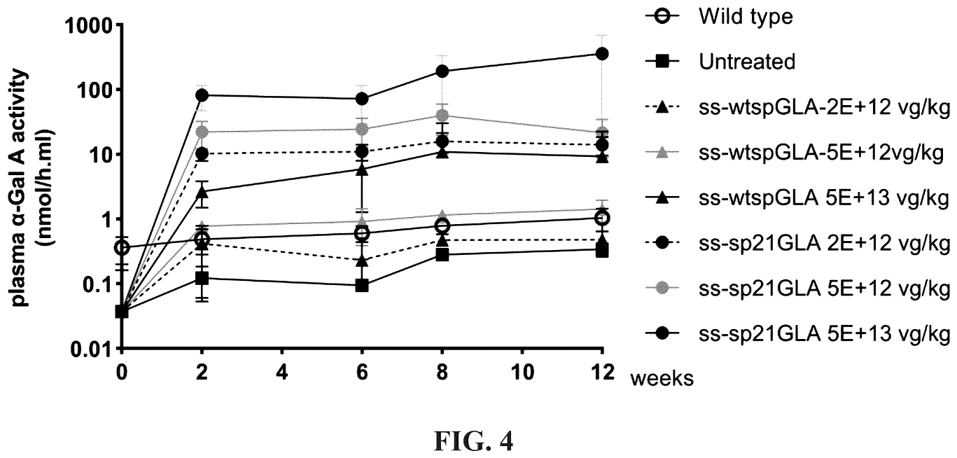

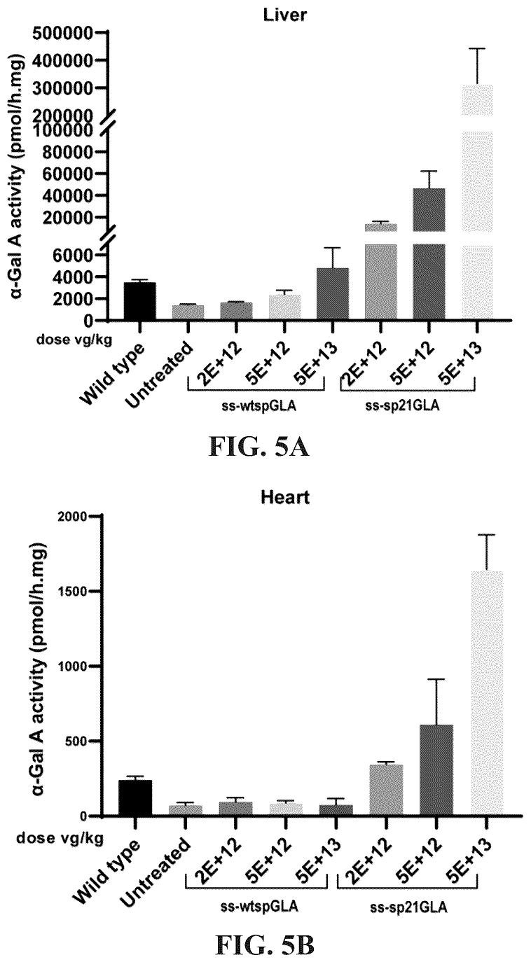

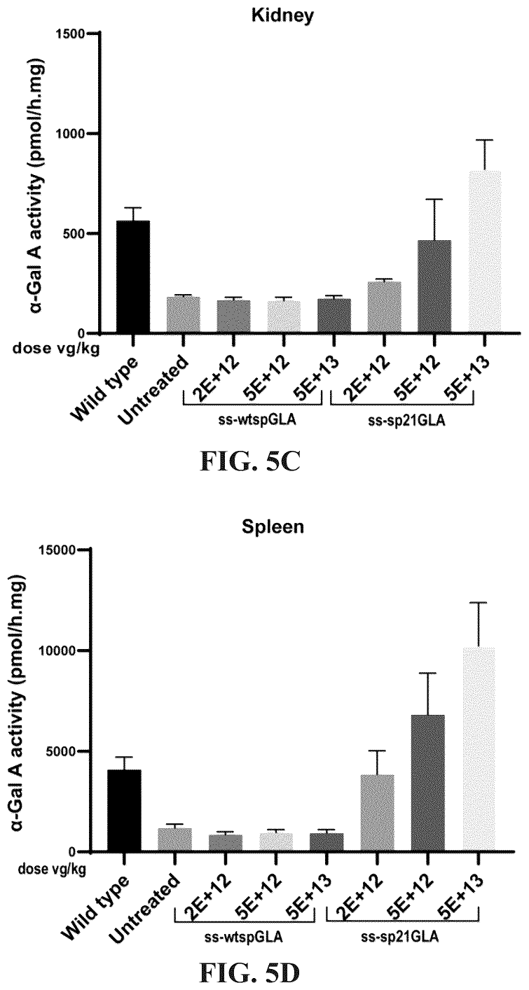

To determine whether α-GAL A fused with heterologous sp similarly increases α-GAL A activity in Fabry mice, Fabry mice were injected with either 2E+12 vg/kg (low dose), 5E+12 vg/kg (medium dose), or 5E+13 vg/kg (high dose) rAAV vector expressing α-GAL A fused to wt sp (ss-wtspGLA), or heterologous sp21 (ss-sp21 GLA). α-GAL A activity was quantified from orbital blood-derived plasma at 0, 2, 6, 8, and 12-weeks post-injection. Compared to Fabry mice control, both vectors produced higher levels of α-GAL A activity (FIG. 4). In all three dose groups, mice administrated ss-sp21GLA exhibited higher levels of α-GAL A activity compared to mice injected ss-wtspGLA (FIG. 4). The highest activity was observed under high dose of ss-sp21 GLA. Similarly, liver, heart, kidney, and spleen tissues were harvested at 12 weeks post injection from mice treated under conditions mentioned previously. The results showed a dose-dependent effect of overall α-GAL A activity levels in various organs in mice injected with ss-sp21 GLA, while in mice treated with ss-wtspGLA, overall α-GAL A activity levels in various organs showed less impact by dose (FIGS. 5A-D for liver, heart, kidney, and spleen tissues, respectively).

### Example 2: rAAV's Expressing α-GAL A Fused with Sp21 Increased α-GAL A Liver, Heart, and Kidney Expression in Fabry Mice

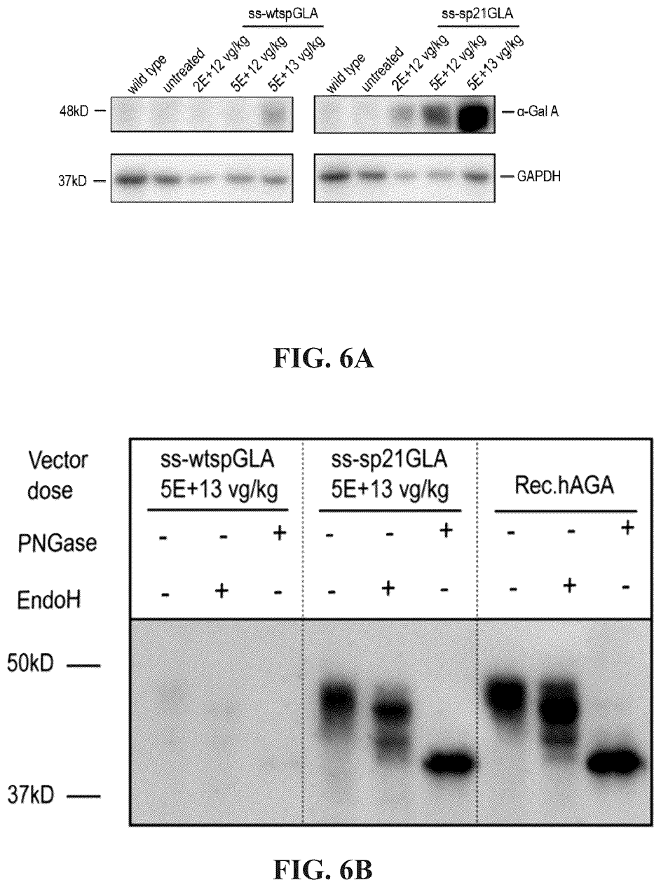

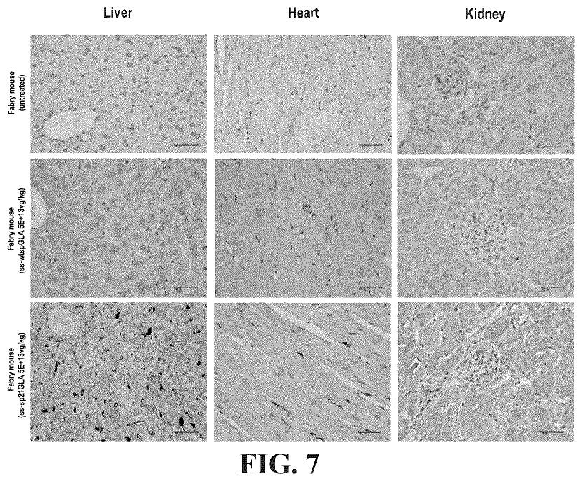

To assess whether ss-sp21GLA administration increases α-GAL A liver expression and produces active form with post-translational modifications, Fabry mice liver samples were examined 12 weeks following injection with low dose, medium dose, or high dose ss-sp21 GLA or ss-wtspGLA. Mice treated with any dose of ss-sp21 GLA displayed higher levels of α-GAL A in the liver, heart, and kidney compared to mice subjected to ss-wtspGLA treatment, as shown in FIG. 6A and FIG. 7. Additionally, glycosylation analysis of liver protein at 12 weeks post injection showed α-GAL A produced by both vectors was glycosylated and processed to the mature form (FIG. 6B). This suggests that ss-sp21 GLA administration increased α-GAL A presence in the liver of Fabry mice compared to ss-wtspGLA, and α-GAL A produced by both vectors was glycosylated and processed to mature form. Recombinant α-GAL A was used as a positive control for glycosylation test in vitro (Rec.hAGA).

### Example 3: rAAV's Expressing α-GAL A Fused with Sp21 Reduced Lyso-Gb3 and May Slow Weight Gain without Perturbing Tissue Structure

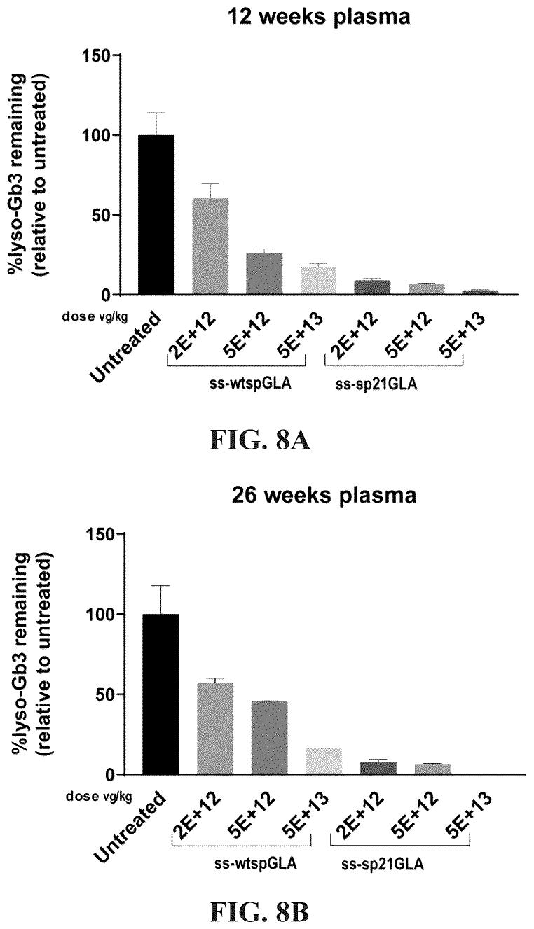

α-GAL A deficiency may lead to progressive and systemic accumulation of sphingolipids, such as globotriaosylceramide (Gb3) and its deacylated derivative, globotriaosylsphingosine (lyso-Gb3). Therefore, restoring α-GAL A may reduce Gb3 and lyso-Gb3 levels. To determine whether ss-sp21 GLA is effective in reducing lyso-Gb3, mice were treated with low, medium, or high dose ss-sp21GLA or ss-wtspGLA. At both 12-weeks (FIG. 8A) and 26-weeks (FIG. 8B) following injection, lyso-Gb3 reduction was greater following injection of ss-sp21GLA at all doses compared to mice injected with ss-wtspGLA. This suggests that ss-sp21 GLA administration showed greater efficacy in lyso-Gb3 reduction relative to ss-wtspGLA administration.

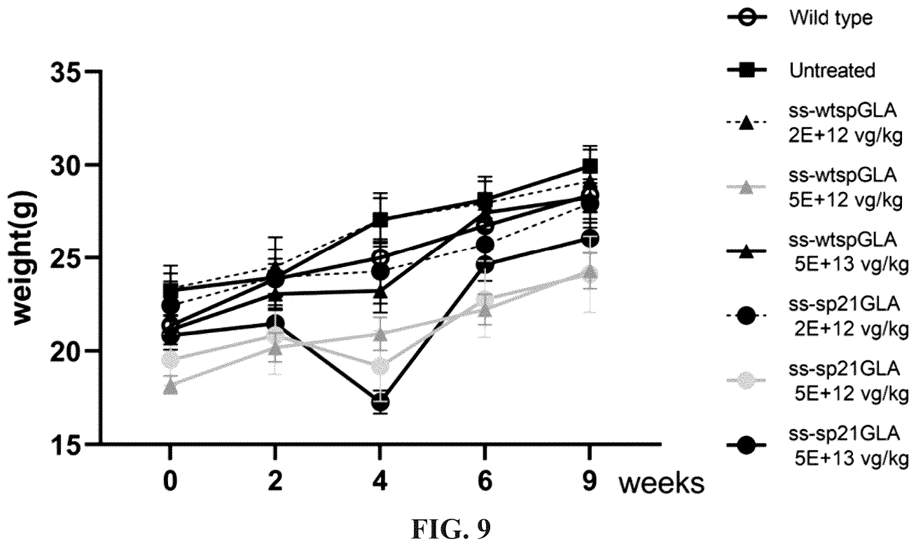

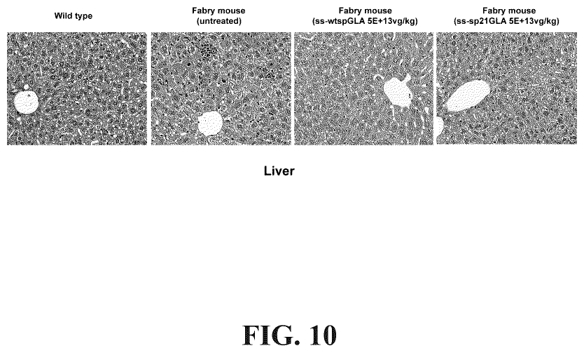

To determine whether ss-sp21GLA administration has an effect on the growth of mice, that is, whether the treatment is safe., mice were treated with low, medium, or high dose ss-sp21 GLA or ss-wtspGLA. Weights were recorded at 0, 2, 4, 6, and 9 weeks. Body weights revealed overall similar velocities of weight gain during growth, but weight of mice injected with a medium or high dose of ss-sp21GLA decreased slightly at 4-weeks post-injection, as well as medium dose of ss-wtspGLA (FIG. 9). This suggests that ss-sp21GLA administration may be effective in slowing weight gain at some dosages.

To determine whether expressing α-GAL A by ss-sp21 GLA administration has negative impacts in liver, tissues were harvested from WT mice, untreated Fabry mice, and Fabry mice treated with high dose ss-wtspGLA or ss-sp21 GLA and assessed by Hematoxylin-Eosin staining (HE). There were no obvious pathological changes in liver of Fabry mice after treatment. This suggests that high dose of ss-sp21 GLA administration was not harmful to the liver.

### Example 4: Functional Variants of the hGLA Nucleotide Sequence Encoding α-GAL A Increased α-GAL A Expression in HepG2 Cells and in Murine Tissues

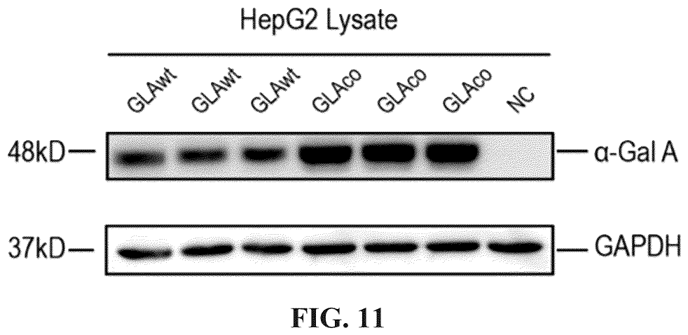

To determine whether a modification of the wt hGLA nucleotide sequence increases α-GAL A expression, plasmids comprising a functional variant of wt SP (wt SP c.o.) and a functional variant of wt hGLA, hGLA c.o.v1 (sometimes “cohGLA”), were transfected into HepG2 cells and protein lysate was assessed for α-GAL A expression. Transfection with the hGLA c.o.v1 nucleotide sequence associated with increased expression of α-GAL A as shown in FIG. 11, suggesting that optimizing the wt hGLA nucleotide sequence to hGLA c.o.v1 can increase α-GAL A expression in cells.

To determine whether sc-rAAV vectors comprising the hGLA c.o.v1 nucleotide sequence could modulate α-GAL A expression and activity in mice, Fabry mice were administered low or medium dose ss-sp21GLA, FD802-1, FD802-2, FD802-3, or FD802-4. Plasma α-GAL A activity was measured at 0, 2, 4, 6, and 8 weeks. FD802-1, 2, 3, and 4 vectors comprising hGLA c.o.v1 nucleotide sequences showed increased α-GAL A activity at all measurements relative to ss-sp21GLA, as shown in FIG. 12. Similarly, FD802-1, 2, 3, and 4 vectors comprising hGLA c.o.v1 nucleotide sequences correlated with increased α-GAL A activity in liver, heart, kidney, and spleen relative to ss-sp21 GLA vectors administered at the same dose, as shown in FIGS. 13 and 14. This suggests that the hGLA c.o.v1 nucleotide sequence increased α-GAL A expression and activity relative to the wt hGLA nucleotide sequence.

### Example 5: Functional Variants of the hGLA Nucleotide Sequence Encoding α-GAL A Increased Liver α-GAL A Expression in Mice and Reduced Lyso-Gb3 without Pathological Changes in Liver

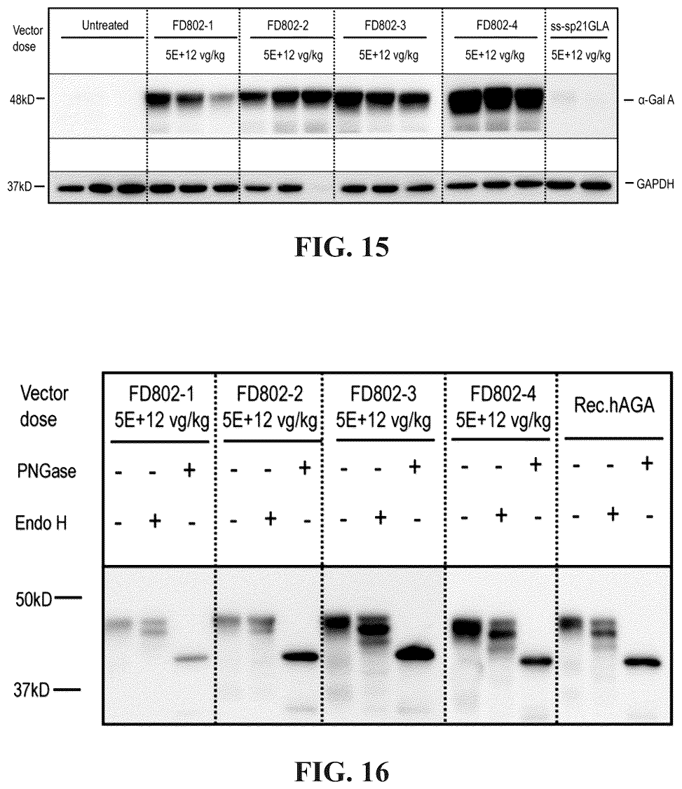

To assess whether the hGLA c.o.v1 nucleotide sequence increases liver α-GAL A expression relative to wt hGLA nucleotide sequences, liver samples of Fabry mice 14 weeks following injection with medium dose FD802-1, FD802-2, FD802-3, FD802-4, and ss-sp21 GLA were examined for α-GAL A expression (FIG. 15). α-GAL A expression levels detected in the liver in mice injected with medium does of FD802-1, FD802-2, FD802-3, or FD802-4 were higher than in mice injected with ss-sp21 GLA, and the highest α-GAL A expression levels were observed in the FD802-4 treated mice (FIG. 15).

Additionally, glycosylation analysis of liver protein at 14 weeks post injection of FD802-1, 2, 3, and 4 vectors comprising hGLA c.o.v1 showed α-GAL A produced by said vectors was glycosylated and processed to the mature form (FIG. 16). Recombinant α-GAL A was used as a positive control for glycosylation test in vitro (Rec.hAGA).

As mentioned previously, α-GAL A deficiency may lead to progressive and systemic accumulation of sphingolipids, such as Gb3 and its deacylated derivative, lyso-Gb3. Therefore, restoring α-GAL A may reduce Gb3 and lyso-Gb3 levels. To determine whether FD802-1, 2, 3, and 4 vectors comprising hGLA c.o.v1 nucleotide sequences are effective in reducing lyso-Gb3, mice were treated with low or medium dose FD802-1, FD802-2, FD802-3, FD802-4, and ss-sp21 GLA. Quantification of lyso-Gb3 in plasma at 6 weeks (FIG. 17A) and 14 weeks (FIG. 17B) post injection by LC-MS/MS confirmed the effective reduction of the substrate by α-GAL A in mice administrated vectors compared to untreated mice, with dose-dependent effect. FD802-1, FD802-2, FD802-3, and FD802-4 led to better Lyso-Gb3 elimination compared to ss-sp21 GLA. Residual substrate levels were less than 5% of untreated control in mice injected with low or medium dose vectors at as early as 6 weeks post injection (FIG. 17A), and the best clearance was reached in FD802-4 treated mice. A higher α-GAL A activity level was produced in mice injected with one of the four sc-AAV8 vectors, FD802-1, FD802-2, FD802-3, and FD802-4.

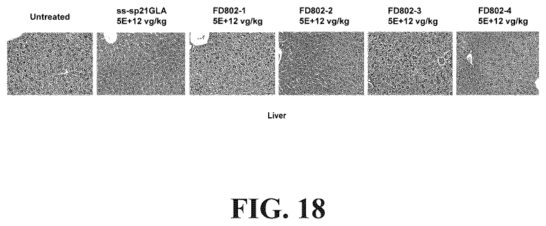

To assess whether administration FD802-1, FD802-2, FD802-3, and FD802-4 vectors comprising an hGLA c.o.v1 nucleotide sequence alter tissue histology, liver tissues were harvested from untreated Fabry mice and Fabry mice treated with medium dose of FD802-1, FD802-2, FD802-3, FD802-4, or ss-sp21GLA. Hematoxylin-eosin (HE) analysis by H&E staining demonstrated that no pathological changes were observed in the liver of Fabry mice treated with FD802-1, 2, 3, and 4 at medium dose, as shown in FIG. 18. This suggests the safety of the treatment in Fabry mice with the administration of FD802-1, FD802-2, FD802-3, FD802-4 vectors comprising the hGLA c.o.v1 nucleotide sequence at a medium dose.

### Example 6: Successful α-GAL A Expression Using Other Vector Serotypes

To determine whether rAAV serotype corresponds with α-GAL A expression and secretion, HepG2 cells were transduced with FD802-4 comprising serotypes rAAV1, rAAV2, . . . , or rAAV10 and α-GAL A activity was measured. Data from WB confirmed α-GAL A expression in (Lysate) and secretion (Media) from HepG2 cells delivered by rAAV encapsidated with various serotype capsids (FIG. 19A). Activity analysis of α-GAL A secreted showed α-GAL A produced by those vectors was processed to the active form (FIG. 19B), with serotype rAAV6 corresponding to the highest α-GAL A activity. The variance of expression and activity level among those vectors may be due to packaging efficiencies. Various serotypes of AAV may transduce α-GAL A expression in human cells for therapeutic use.

### Example 7: α-GAL A Fused with TAT p47-57 Did not Alter Activity of Secreted α-GAL A

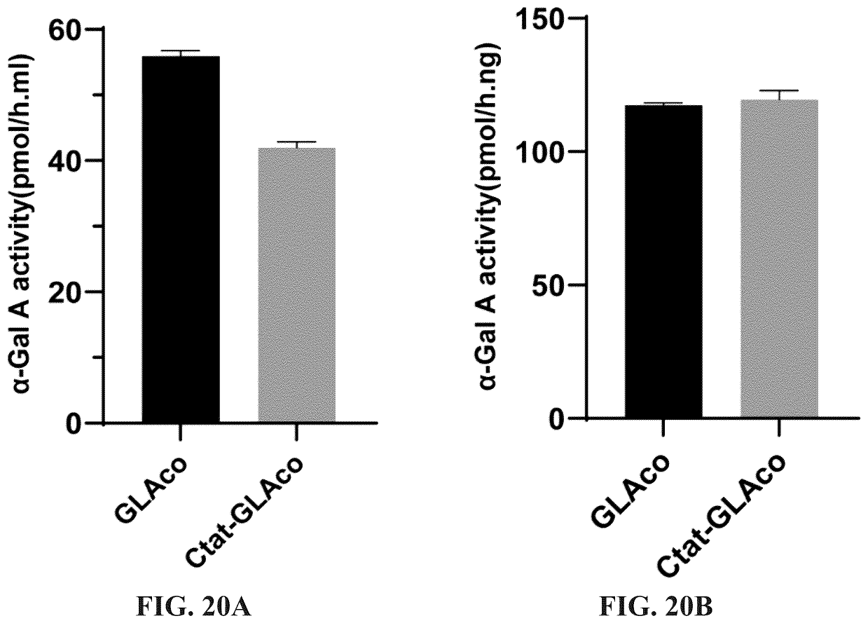

To determine whether incorporating nucleotide sequences for TAT p47-57, a cell-penetrating peptide, in an rAAV vector comprising an hGLA nucleotide sequence could improve α-GAL A activity, HepG2 cells were transfected with a plasmid comprising TAT nucleotide sequence (tat, which is the wild type nucleotide sequence expressing TAT p47-57), wtSP c.o. and hGLA c.o.v1, as well as a plasmid comprising hGLA c.o.v1 and wtSP c.o., and activity of α-GAL A secreted were quantified. α-GAL A activity quantification using culture media revealed that hGLA fused with TAT p47-57 (Ctat-GLAco) producing α-GAL A with a lower activity level compared to hGLA not fused with TAT p47-57 (GLAco) (FIG. 20A), while α-GAL A activity quantification using an equal amount of α-GAL A produced by the two vectors showed no differences between the two vectors (FIG. 20B), indicating fusion of TAT p47-57 with hGLA decreased transgene expression or secretion, but did not affect α-GAL A activity.

### Example 8: α-GAL A Fused with TAT p47-57 May Increase α-GAL A Tissue Permeability without Altering Expression or Glycosylation of α-GAL A Nor Lyso-Gb3 Clearance

To determine whether α-GAL A fused with TAT p47-57 increases α-GAL A activity relative to α-GAL A not fused with TAT, Fabry mice were administered with either a low or medium dose of FD802-3 or FD802-5, where FD802-5 comprised TAT nucleotide sequences, as shown in FIG. 1B. Mice injected with FD802-3 or FD802-5 vectors expressing TAT p47-57 demonstrated higher levels of α-GAL A activity relative to untreated Fabry mice, while FD802-5 vectors showed dose-dependent α-GAL A activity levels, as shown in FIG. 21A. Similarly, α-GAL A activity levels were measured in the liver, heart, kidney, and spleen of Fabry mice 14 weeks post-injection with low or medium dose of FD802-3 or FD802-5. Slightly decrease of α-GAL A activity in the liver was observed, but slightly increased α-GAL A activity level in the heart and kidney and were observed in mice injected with the vector expressing TAT p47-57 (FD802-5) (FIG. 21B). Immunohistochemical data from tissue samples at 14 weeks post injection confirmed the expression of α-GAL A in the liver of mice administrated medium dose of vectors. Improved α-GAL A protein levels were observed in the heart in Fabry mice injected with FD802-5 compared to FD802-3 injected Fabry mice (FIG. 22). This suggests tissue permeability of α-GAL A may be improved in some tissue types with administration of vectors expressing α-GAL A fused with TAT p47-57.

To determine whether fusion of TAT p47-57 to α-GAL A alters liver expression or glycosylation of α-GAL A, liver samples of Fabry mice 14-weeks following injection with medium dose of FD802-3 or FD802-5 were assessed for expression and glycosylation of α-GAL A. Mice injected with FD802-3 or FD802-5 vectors demonstrated increased expression of α-GAL A compared to untreated mice, as shown in FIG. 23A. However, there was no visible difference in expression between the vectors which expressed TAT peptide, and those that did not. Glycosylation analysis of liver protein at 14 weeks pi showed α-GAL A produced by both vectors was glycosylated and was processed to the active form (FIG. 23B). This suggests fusion of TAT p47-57 to α-GAL A did not hinder the expression or glycosylation of α-GAL A.

To determine whether fusion of TAT p47-57 to α-GAL A is effective in reducing lyso-Gb3, mice were treated with low or medium dose FD802-3 and FD802-5, and were assessed for lyso-Gb3 levels. At both 6-weeks (FIG. 24A) and 14-weeks (FIG. 24B) following injection, plasma lyso-Gb3 reduced with administration of all FD802-3 and FD802-5, with FD802-5 showing a dose-dependent effect. Medium dose of FD802-5 injection led to similar lyso-Gb3 elimination compared to FD802-3. Residual substrate levels were less than 5% of PBS control in mice injected with low or medium dose vectors at as early as 6 weeks post injection (FIG. 24A).

Liver tissues of Fabry mice treated with medium dose of FD802-3, or FD802-5 were assessed using H&E staining. The administration showed no observable negative effects on the liver, as shown in FIG. 25. This suggests the safety of the treatment in Fabry mice as α-GAL A fused with TAT p47-57 did not alter tissue integrity when administered.

### Example 9

### 1.Clinical Dosage

(1) Patients with Fabry disease were recruited for an investigator-initiated clinical study (IIT). rAAV expressing α-GAL A fused to sp21 was administered to adult male patients with Classic Fabry disease at a single intravenous dose of 1×1013 vg/kg, the dose group was planned to enroll 1-3 patients, with 2 actually enrolled to date. The enzyme activity is tested weekly from 1 to 8 weeks, every 2 weeks from 8 to 12 weeks, every 4 weeks from 12 to 32 weeks, and once at weeks 40 and 52 after administration, when patients entered the long-term follow-up period, the test was performed once at each of the 1.5/2/3/4/5 years after drug administration, respectively. After 1 week of administration, the plasma enzyme activity of both subjects had exceeded the normal value level, and the highest value had reached 3.87 times to 15.74 times of the plasma enzyme activity level of normal subjects, and remained stable. Which did effectively remove the substrate Gb3 in plasma, the plasma levels of Gb-3 in 2 patients were decreased by 56.22% and 44.90% at 16 and 8 weeks after administration, respectively.

(2) rAAV expressing α-GAL A fused to sp21 will enroll 1-3 subjects in the 3×1013 vg/kg dose group; no subjects are currently enrolled.

1. An engineered peptide comprising or consisting of a portion of human alphα-galactosidase A (α-GAL A) or a functional variant thereof, and a portion of signal peptide of tissue plasminogen activator (sp21, sp18, sp20 or sp22) or α-GAL A (wt sp)-, or a functional variant thereof.

2. The engineered peptide of claim 1, further comprising a portion of a cell-penetrating peptide or a functional variant thereof.

3. (canceled)

4. The engineered peptide of claim 1, wherein the portion of α-GAL A or the functional variant thereof comprises a peptide sequence with at least 80%, 85%, 90%, 95%, 99%, or 100% identity to SEQ ID NO: 27.

5. The engineered peptide of claim 1, wherein the portion of sp21, sp18, sp20 or sp22 or the functional variant thereof comprises a peptide sequence with at least 80%, 85%, 90%, 95%, 99%, or 100% identity to SEQ ID NOs: 22, 19, 21, or 23.

6. The engineered peptide of claim 2, wherein the portion of cell-penetrating peptide or the functional variant thereof comprises a peptide sequence with at least 80%, 85%, 90%, 95%, 99%, or 100% identity to SEQ ID NOs: 55-63.

7. The engineered peptide of claim 1, wherein the portion of sp21 or the functional variant thereof is fused to the N terminus of the portion of α-GAL A or the functional variant thereof.

8. The engineered peptide of claim 2, wherein the portion of the cell-penetrating peptide or the functional variant thereof is fused to the C terminus of the portion of α-GAL A or the functional variant thereof.

9. A viral vector comprising a nucleotide sequence encoding the engineered peptide of claim 1.

10. (canceled)

11. A viral vector comprising an expression cassette comprising or consisting of a first nucleotide sequence encoding the portion of α-GAL A or the functional variant thereof, a second nucleotide sequence encoding the portion of sp21 or wt sp, or the functional variant thereof, and optionally a third nucleotide sequence encoding the portion of the cell-penetrating peptide or the functional variant thereof, and a promoter.

12. (canceled)

13. The viral vector of claim 11, wherein the second nucleotide sequence is 5′ to the first nucleotide sequence and/or wherein the third nucleotide sequence is 3′ to the first nucleotide sequence.

14. (canceled)

15. The viral vector of claim 9, where the nucleotide sequence encoding the portion of α-GAL A or the functional variant thereof has a nucleotide sequence with at least 80%, 85%, 90%, 95%, 99%, or 100% identity to SEQ ID NO: 25 or 26.

16. The viral vector of claim 9, where the nucleotide sequence encoding the portion of sp21, sp18, sp20 or sp22 or the functional variant thereof has a nucleotide sequence with at least 80%, 85%, 90%, 95%, 99%, or 100% identity to SEQ ID NOs: 22, 19, 21, or 23.

17. The viral vector of claim 9, where the nucleotide sequence encoding the portion of wt sp or the functional variant thereof has a nucleotide sequence with at least 80%, 85%, 90%, 95%, 99%, or 100% identity to SEQ ID NO: 51.

18. The viral vector of claim 9, where the nucleotide sequence encoding the portion of cell-penetrating peptide or the functional variant thereof has a nucleotide sequence with at least 80%, 85%, 90%, 95%, 99%, or 100% identity to SEQ ID NO: 24, 52, 53, or 54.

19. (canceled)

20. (canceled)

21. (canceled)

22. (canceled)

23. (canceled)

24. A method of treating Fabry disease or reducing one or more symptoms of Fabry disease in a subject, comprising administering the engineered peptide of claim 1, a viral vector comprising a nucleotide sequence encoding the engineered peptide of claim 1, or a pharmaceutical composition comprising the engineered peptide of claim 1 or the viral vector to the subject.

25. A method of increasing α-Gal A expression in one or more target tissues of a subject, comprising administering the engineered peptide of claim 1, a viral vector comprising a nucleotide sequence encoding the engineered peptide of claim 1, or a pharmaceutical composition comprising the engineered peptide of claim 1 or the viral vector of to the subject.

26. A method of reducing sphingolipid storage in one or more target tissues of a subject, comprising administering the engineered peptide of claim 1, a viral vector comprising a nucleotide sequence encoding the engineered peptide of claim 1, or a pharmaceutical composition comprising the engineered peptide of claim 1 or the viral vector to the subject.

27. (canceled)

28. (canceled)

29. The method of claim 24, wherein the engineered peptide, the viral vector, or the pharmaceutical composition comprising the engineered peptide or the viral vector is administered to the subject.

30. The method of claim 24, wherein the engineered peptide, the viral vector, or the pharmaceutical composition comprising the engineered peptide or the viral vector is administered to the subject once every 12 months.

31. (canceled)

32. The method of claim 24, wherein the viral vector or the pharmaceutical composition comprising the viral vector is administered to the subject in one or more doses of 0.5E+10 vg/kg to 7E+14 vg/kg.


SEQUENCE LISTING (66 sequences)

SEQ ID NO: 1 - DNA, 93 bp, Synthetic construct
  source 1..93: mol_type = other DNA
atgcagctgaggaacccagaactacatctgggctgcgcgcttgcgcttcgcttcctggccctcgtttcctgggacatccctggggctagagca

SEQ ID NO: 2 - DNA, 72 bp, Synthetic construct
  source 1..72: mol_type = other DNA
atgccgtcttctgtctcgtggggcatcctcctgctggcaggcctgtgctgcctggtccctgtctccctggct

SEQ ID NO: 3 - DNA, 45 bp, Synthetic construct
  source 1..45: mol_type = other DNA
atgaatctacttctgatccttacctttgttgcagctgctgttgct

SEQ ID NO: 4 - DNA, 54 bp, Synthetic construct
  source 1..54: mol_type = other DNA
atggctttcctctggctcctctcctgctgggccctcctgggtaccaccttcggc

SEQ ID NO: 5 - DNA, 66 bp, Synthetic construct
  source 1..66: mol_type = other DNA
atggcctccaggctgaccctgctgaccctcctgctgctgctgctggctggggatagagcctcctca

SEQ ID NO: 6 - DNA, 60 bp, Synthetic construct
  source 1..60: mol_type = other DNA
atgtacaggatgcaactcctgtcttgcattgcactaagtcttgcacttgtcacaaacagt

SEQ ID NO: 7 - DNA, 54 bp, Synthetic construct
  source 1..54: mol_type = other DNA
atgaagtgggtaacctttatttcccttctttttctctttagctcggcttattcc

SEQ ID NO: 8 - DNA, 48 bp, Synthetic construct
  source 1..48: mol_type = other DNA
atgctgctgttgtcagcgcttctccttgggcttgcccatgggtactcc

SEQ ID NO: 9 - DNA, 48 bp, Synthetic construct
  source 1..48: mol_type = other DNA
atgctgctgttgtcagcgcttctccttgggcttgcctttgggtactcc

SEQ ID NO: 10 - DNA, 48 bp, Synthetic construct
  source 1..48: mol_type = other DNA
atgctgctgttgtcagcgcttctccttgggcttgcctgtgggtactcc

SEQ ID NO: 11 - DNA, 48 bp, Synthetic construct
  source 1..48: mol_type = other DNA
atgctgctgagtttcgcgcttctccttgggcttgcccttgggtactcc

SEQ ID NO: 12 - DNA, 48 bp, Synthetic construct
  source 1..48: mol_type = other DNA
atgctgctggttttagcgcttctccttgggcttgcccttgggtactcc

SEQ ID NO: 13 - DNA, 48 bp, Synthetic construct
  source 1..48: mol_type = other DNA
atgctgctgttgtcagcgcttctccttgggcttgccatggggtactcc

SEQ ID NO: 14 - DNA, 48 bp, Synthetic construct
  source 1..48: mol_type = other DNA
atgctgctgagaatagcgcttctccttgggcttgcccatgggtactcc

SEQ ID NO: 15 - DNA, 48 bp, Synthetic construct
  source 1..48: mol_type = other DNA
atgctgctgttgtcagcgcttctccttgggcttgccattgggtactcc

SEQ ID NO: 16 - DNA, 48 bp, Synthetic construct
  source 1..48: mol_type = other DNA
atgctgctgttgccagcgcttctccttgggcttgcccatgggtactcc

SEQ ID NO: 17 - DNA, 48 bp, Synthetic construct
  source 1..48: mol_type = other DNA
atgctgctggtgccagcgcttctccttgggcttgcccatgggtactcc

SEQ ID NO: 18 - DNA, 48 bp, Synthetic construct
  source 1..48: mol_type = other DNA
atgctgctggagcacgcgcttctccttgggcttgcccatgggtactcc

SEQ ID NO: 19 - DNA, 51 bp, Synthetic construct
  source 1..51: mol_type = other DNA
atgggagtgaaagttctttttgcccttatttgtattgctgtggccgaggcc

SEQ ID NO: 20 - DNA, 51 bp, Synthetic construct
  source 1..51: mol_type = other DNA
atggatataaaggttgtctttactcttgttttctcagcattggttcaggca

SEQ ID NO: 21 - DNA, 54 bp, Synthetic construct
  source 1..54: mol_type = other DNA
atgaagataataattctgtctgttatattggcctactgtgtcaccgtcaactgt

SEQ ID NO: 22 - DNA, 66 bp, Synthetic construct
  source 1..66: mol_type = other DNA
atggatgcaatgaagagagggctctgctgtgtgctgctgctgtgtggagcagtcttcgtttcggca

SEQ ID NO: 23 - DNA, 57 bp, Synthetic construct
  source 1..57: mol_type = other DNA
atgggagtgaaagttctttttgcccttatttgtattgctgtggccgaggtgacggga

SEQ ID NO: 24 - DNA, 33 bp, Synthetic construct
  source 1..33: mol_type = other DNA
tatggcaggaagaagcggagacagcgacgaaga

SEQ ID NO: 25 - DNA, 1197 bp, Synthetic construct
  source 1..1197: mol_type = other DNA
ctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 26 - DNA, 1197 bp, Synthetic construct
  source 1..1197: mol_type = other DNA
ctggacaacggcctggccagaacccccaccatgggctggctgcactgggagagattcatgtgcaacctggactgccaggaggagcccgacagctgcatctccgagaagctgttcatggagatggccgagctgatggtctccgagggctggaaggacgccggctatgagtacctgtgcattgatgactgctggatggcccctcaaagagactctgagggcagactgcaggccgacccccagagattccctcacggcatcagacagctggccaactacgtgcacagcaagggcctgaagctgggcatctacgccgatgtgggcaataagacctgtgccggatttcctggatcttttggctactatgacatcgatgcccagacctttgccgactggggagtggacctgctgaagtttgatggatgctactgtgacagcctggagaacctggccgacggctacaagcacatgagcctggccctgaatagaacaggcagaagcatcgtgtacagctgcgagtggcccctgtatatgtggcccttccaaaagcctaactacaccgagatcaggcagtactgcaaccactggagaaacttcgccgacatcgatgacagctggaagagcatcaagagcatcctggactggacaagcttcaaccaggagagaatcgtggatgtggccggccctggcggctggaatgaccctgacatgctggtcattggcaacttcggcctgagctggaaccagcaggtgacacagatggccctgtgggccatcatggccgctcccctgttcatgagcaacgacctgagacacatcagcccacaagccaaggccctgctacaagacaaggacgtgatcgccatcaatcaagaccctctgggaaagcagggctaccagctgagacagggcgacaacttcgaagtgtgggagagacctctgagcggactggcctgggccgtggccatgatcaacagacaggagatcggcggccccagaagctacaccatcgctgtggcctctctgggcaagggcgtggcctgcaacccagcctgcttcatcacacaactgctgcctgtgaagagaaagctgggcttctacgagtggaccagcagactgagaagccacatcaaccccaccggcaccgtgctgctgcaactggagaacaccatgcagatgagcctgaaggacctgctgtga

SEQ ID NO: 27 - protein, 398 aa, Synthetic construct
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 28 - protein, 31 aa, Synthetic construct
MQLRNPELHLGCALALRFLALVSWDIPGARA

SEQ ID NO: 29 - protein, 24 aa, Synthetic construct
MPSSVSWGILLLAGLCCLVPVSLA

SEQ ID NO: 30 - protein, 15 aa, Synthetic construct
MNLLLILTFVAAAVA

SEQ ID NO: 31 - protein, 18 aa, Synthetic construct
MAFLWLLSCWALLGTTFG

SEQ ID NO: 32 - protein, 22 aa, Synthetic construct
MASRLTLLTLLLLLLAGDRASS

SEQ ID NO: 33 - protein, 20 aa, Synthetic construct
MYRMQLLSCIALSLALVTNS

SEQ ID NO: 34 - protein, 18 aa, Synthetic construct
MKWVTFISLLFLFSSAYS

SEQ ID NO: 35 - protein, 16 aa, Synthetic construct
MLLLSALLLGLAHGYS

SEQ ID NO: 36 - protein, 16 aa, Synthetic construct
MLLLSALLLGLAFGYS

SEQ ID NO: 37 - protein, 16 aa, Synthetic construct
MLLLSALLLGLACGYS

SEQ ID NO: 38 - protein, 16 aa, Synthetic construct
MLLSFALLLGLALGYS

SEQ ID NO: 39 - protein, 16 aa, Synthetic construct
MLLVLALLLGLALGYS

SEQ ID NO: 40 - protein, 16 aa, Synthetic construct
MLLLSALLLGLAMGYS

SEQ ID NO: 41 - protein, 16 aa, Synthetic construct
MLLRIALLLGLAHGYS

SEQ ID NO: 42 - protein, 16 aa, Synthetic construct
MLLLSALLLGLAIGYS

SEQ ID NO: 43 - protein, 16 aa, Synthetic construct
MLLLPALLLGLAHGYS

SEQ ID NO: 44 - protein, 16 aa, Synthetic construct
MLLVPALLLGLAHGYS

SEQ ID NO: 45 - protein, 16 aa, Synthetic construct
MLLEHALLLGLAHGYS

SEQ ID NO: 46 - protein, 17 aa, Synthetic construct
MGVKVLFALICIAVAEA

SEQ ID NO: 47 - protein, 17 aa, Synthetic construct
MDIKVVFTLVFSALVQA

SEQ ID NO: 48 - protein, 18 aa, Synthetic construct
MKIIILSVILAYCVTVNC

SEQ ID NO: 49 - protein, 22 aa, Synthetic construct
MDAMKRGLCCVLLLCGAVFVSA

SEQ ID NO: 50 - protein, 19 aa, Synthetic construct
MGVKVLFALICIAVAEVTG

SEQ ID NO: 51 - DNA, 93 bp, Synthetic construct
  source 1..93: mol_type = other DNA
atgcagctgagaaaccccgagctgcacctgggctgtgccctggccctgcgctttctggccctggtgagttgggatatccctggcgccagagcc

SEQ ID NO: 52 - DNA, 39 bp, Synthetic construct
  source 1..39: mol_type = other DNA
ggcaggaagaagcggagacagcgacgaagacctcctcaa

SEQ ID NO: 53 - DNA, 48 bp, Synthetic construct
  source 1..48: mol_type = other DNA
cgccagataaagatttggttccagaatcggcgcatgaagtggaagaag

SEQ ID NO: 54 - DNA, 72 bp, Synthetic construct
  source 1..72: mol_type = other DNA
ctgggcacatacacgcaggacttcaacaagtttcacacgttcccccaaactgcaattggggttggagcacct

SEQ ID NO: 55 - protein, 11 aa, Synthetic construct
YGRKKRRQRRR

SEQ ID NO: 56 - protein, 13 aa, Synthetic construct
GRKKRRQRRRPPQ

SEQ ID NO: 57 - protein, 16 aa, Synthetic construct
RQIKIWFQNRRMKWKK

SEQ ID NO: 58 - protein, 24 aa, Synthetic construct
LGTYTQDFNKFHTFPQTAIGVGAP

SEQ ID NO: 59 - protein, 9 aa, Synthetic construct
RRRRRRRRR

SEQ ID NO: 60 - protein, 18 aa, Synthetic construct
LLIILRRRIRKQAHAHSK

SEQ ID NO: 61 - protein, 21 aa, Synthetic construct
KETWWETWWTEWSQPKKKRKV

SEQ ID NO: 62 - protein, 27 aa, Synthetic construct
GWTLNSAGYLLGKINLKALAALAKKIL

SEQ ID NO: 63 - protein, 18 aa, Synthetic construct
KLALKLALKALKAALKLA

SEQ ID NO: 64 - DNA, 93 bp, Synthetic construct
  source 1..93: mol_type = other DNA
atgcaactgagaaatcctgaactccacctgggatgtgccctggctctgagatttctggccctggtgtcttgggacatccctggtgctagagcc

SEQ ID NO: 65 - DNA, 93 bp, Synthetic construct
  source 1..93: mol_type = other DNA
atgcagctgagaaatcctgagctgcacctgggctgtgccctggctctgagatttctggctctggtgtcctgggacatccctggtgctagagcc

SEQ ID NO: 66 - DNA, 93 bp, Synthetic construct; identical to SEQ ID NO: 1
  source 1..93: mol_type = other DNA